(19) United States
(12) Patent Application Publication
(10) Pub. No.: US 2011/0038921 A1
(43) Pub. Date: Feb. 17, 2011

(54) METHODS AND COMPOSITIONS FOR TEMPORAL RELEASE OF AGENTS FROM A BIODEGRADABLE SCAFFOLD

(75) Inventors: Xuejun Wen, Mount Pleasant, SC (US); Keith L. Kirkwood, Mount Pleasant, SC (US)

Correspondence Address: MYERS BIGEL SIBLEY & SAJOVEC, PO BOX 37428, RALEIGH, NC, 27627, US

(73) Assignee: Clemson University Research Foundation

(21) Appl. No.: 12/856,299

(22) Filed: Aug. 13, 2010

Related U.S. Application Data
(60) Provisional application No. 61/233,535, filed on Aug. 13, 2009.

(51) Int. Cl. A61K 9/70; A61K 9/00; A61K 38/00; A61K 31/351; A61K 38/22; A61K 38/18; A61K 38/20; A61K 38/30; A61P 1/02; A61P 17/02

(52) U.S. Cl. 424/445; 424/400; 514/2.3; 514/460; 514/9.5; 514/7.6; 514/9.7; 514/8.2; 514/8.9; 514/9.6; 514/9.1; 514/9.2; 514/8.5; 514/8.6; 514/8.8; 424/85.2; 514/8.7

(57) ABSTRACT

The present invention provides methods and compositions for sequentially and separately reducing infection and/or inflammation and regenerating tissue at a lesion site, by contacting the lesion site with a biodegradable scaffold that first delivers one or more agents at the lesion site to reduce infection and/or inflammation and then delivers one or more agents to regenerate tissue at the lesion site after inflammation is reduced.

### STATEMENT OF PRIORITY

This application claims the benefit, under 35 U.S.C. §119(e) of U.S. Provisional Application Ser. No. 61/233,535, filed Aug. 13, 2009, the entire contents of which are incorporated herein by reference.

### FIELD OF THE INVENTION

The present invention relates to compositions and methods for temporal release of agents from a biodegradable scaffold.

### BACKGROUND OF THE INVENTION

Periodontal disease is characterized by inflammation and destruction of supporting alveolar bone and periodontal tissues. Lipopolysaccharide (LPS) from Gram-negative bacteria present in the oral biofilm is the major microbial antigen activating innate and acquired immunity, generating inflammatory responses that can result in the destruction of the periodontium. Thus, attenuating LPS-elicited inflammatory responses is needed to decrease inflammation, permitting subsequent regenerative therapies.

The p38 mitogen-activated protein kinase (MAPK) has been shown to be a major signaling pathway needed to mediate LPS-induced periodontal tissue loss. The tissue preservation observed with p38 inhibitors was due to the decrease in the production of inflammatory cytokines at the post-transcriptional level, leading towards suppression of osteoclastogenesis. Studies have also shown that the (ERK) MAPK pathway is needed, in addition to p38 MAPK, for LPS-stimulated matrix metalloproteinases (MMPs) and other proinflammatory cytokines in mononuclear cells.

Furthermore, biodegradable scaffolds with nanothickness layer-by-layer (nanoLbL) drug coating have been shown to be effective in the delivery of 1) both p38 and ERK inhibitors that can attenuate LPS-elicited inflammatory responses, and 2) different growth factors [e.g., bone morphogenetic protein 2 (BMP-2); platelet derived growth factor (PDGF)] that can promote cell migration, growth, proliferation and/or differentiation to regenerate the lost periodontal tissue. BMP-2 is known to promote bone regeneration. PDGF is known to support periodontal tissue and skin tissue regeneration through the modulation of chemotaxis, proliferation and differentiation of pluripotent cells. Owing to rapid clearance in vivo and the inability to maintain a therapeutic concentration of growth factors, a local long-term delivery strategy would be ideal for a growth factor-based therapy. In addition, without control of inflammation, regeneration will not be successful. However, with anti-inflammation treatment, the regeneration process may be compromised because signaling through p38 and ERK pathways is required for growth factor induced regeneration. Therefore, an ideal delivery scheme would first promote inflammation resolution with short-term delivery of anti-inflammatory agents (e.g., p38 and/or ERK inhibitors) and then delivery of biomolecules (e.g., growth factors, etc.) for regeneration.

Diabetic ulcers are the most common foot injuries leading to lower extremity amputation. There are several treatment options: 1) increasing blood supply (angioplasty, stent insertion, atherectomy, laser recanalization) to wound area; 2) debridement (e.g., necrotic tissue removal to enhance healing); 3) pressure relief (mechanical therapy, such as total contact casting); 4) infection/bioburden control (chronic wounds are known to exist along a bacterial continuum which ranges from contamination to infection); 5) moist wound healing (topical applications); 6) physical modalities (negative pressure wound therapy, e.g., WOUND VAC therapy), electrical stimulation, magnetic therapy); 6) wound environment manipulators (PROMOGRAN) and oxygen therapy (hyperbaric oxygen, topical oxygen); and 7) active methods of healing, such as: a) stimulation of more rapid wound healing by accelerated angiogenesis, stimulation of growth factor release, providing wound matrix for cellular ingrowth, production of required proteins/growth factors; b) platelet-derived growth factor (BECAPLERMIN) application; and c) living human dermal substitutes (e.g., APLIGRAF, DERMAGRAFT).

Various growth factors have been tested, including REGRANEX (PDGF-B)/platelet releasates; epidermal growth factor (EGF), fibroblast growth factor (bFGF), granulocyte macrophage colony stimulating factor (GM-CSF), keratinocyte growth factor-2 (KGF-2) and transforming growth factor beta (TGF-β). A number of recombinant growth factors have been tested in clinical trials as wound healing agents but none have shown consistent clinical results nor been approved for therapeutic use except for PDGF-BB. Despite use of optimal therapy, diabetic ulcers require an average of 4-6 months of treatment to heal. Many patients cannot tolerate the requirements of treatment for 4-6 months or more. Cost in terms of lost productivity, impact on work, exercise and lifestyle is high. Barriers to diabetic wound healing include the inability to control the local environment. The microenvironment in the ulcer is very complicated and includes a biofilm that is resistant to antibiotic treatment and products of bacteria (e.g., endotoxin, such as lipopolysaccharide (LPS)). LPS can cause inflammation (IL-1, IL-6, TNF-alpha up-regulation) and matrix metalloproteinase (MMP) up-regulation, which breaks down the extracellular matrix (tissue). LPS can also inhibit tissue regeneration (MMP 2, 8 and 9 levels are elevated in venous ulcer exudate and are reduced in healing wounds) and cause down-expression of growth factors for diabetic ulcers, such as TGF-beta, PDGF, etc.

As noted above, similar pathology is observed for periodontal disease. Periodontal disease is characterized by inflammation and destruction of supporting alveolar bone and periodontal tissues.

Thus, attenuating LPS-elicited inflammatory responses is needed to decrease inflammation, permitting subsequent regenerative therapies for both periodontal diseases and diabetic ulcers.

Sequential delivery of molecules that can reduce inflammation and molecules that can promote regeneration will be an effective therapeutic approach in disorders such as diabetic ulcers and periodontal disease. Thus, the present invention provides a unique biodegradable scaffold with nano-layer-by-layer (nanoLbL) coatings of different agents at different layers, allowing sequentially controlled delivery of such agents (e.g., anti-inflammatory agents and regenerative agents) from the same scaffold.

### SUMMARY OF THE INVENTION

The present invention provides a biocompatible, biodegradable, three-dimensional scaffold having a surface and an interior, said scaffold comprising: a) a multiplicity of layers, wherein the layers comprise materials that degrade at different rates, with layers at the surface of the scaffold degrading prior to layers at the interior of the scaffold; b) one or more than one first bioactive agent located at the surface of the scaffold; and c) one or more than one second bioactive agent located at the interior of the scaffold, wherein when the scaffold is exposed to an environment surrounding the scaffold, the one or more than one first bioactive agent is released into the environment prior to release of the one or more than one second bioactive agent and following release of the one or more than one first bioactive agent and degradation of the layers at the surface of the scaffold, the one or more than one second bioactive agent is released into the environment, thereby sequentially and separately releasing the one or more than one first bioactive agent and the one or more than one second bioactive agent into the environment.

In the scaffold of this invention, a first bioactive agent can be but is not limited to an antibiotic, an antimicrobial peptide, an antimicrobial agent, an inhibitor of interleukin-1 (IL-1), an inhibitor of interleukin-6 (IL-6), an inhibitor of tumor necrosis factor alpha (TNF-α), an inhibitor of matrix metalloproteinase (MMP) 1, 2, 8 and/or 9, an inhibitor of p38 mitogen activated protein kinase (MAPK), an inhibitor of extracellular signal-related kinase (ERK) (ERK1; ERK2), SBR203580 (p38 inhibitor), PD98059 (ERK inhibitor), U0126 (inhibitor of MMP expression) simvastatin (inhibitor of MMP-1 expression), an anti-inflammatory agent and any combination thereof.

In particular embodiments of the scaffold of this invention, the one or more than first bioactive agent at the surface of the scaffold comprises one or more antimicrobial agents and one or more anti-inflammatory agents. Furthermore, in the scaffold of this invention, the one or more antimicrobial agents can be present in one or more layers at the surface of the scaffold (e.g., in an outer layer at the surface) and the one or more anti-inflammatory agents can be present in one or more layers at the surface of the scaffold (e.g., in an inner layer at the surface, wherein the layers comprise materials that degrade at different rates, thereby sequentially and separately releasing the one or more antimicrobial agent and the one or more anti-inflammatory agent into the environment.

In the scaffold of this invention, the second bioactive agent can be but is not limited to hepatocyte growth factor (HGF) (HGF-1), stromal cell-derived factor (SDF-1), transforming growth factor beta (TGF-β; TGF-β1, TGF-β3), platelet derived growth factor (PDGF) (e.g., Becaplermin; REGRANEX®, PDGF-BB), platelet releasate, epidermal growth factor (EGF), fibroblast growth factor (FGF) (FGF-2), granulocyte macrophage colony stimulating factor (GM-CSF), keratinocyte growth factor-2 (KGF-2), insulin-like growth factor (IGF) (IGF-I, IGF-II), bone morphogenetic protein (BMP) (BMP-2, BMP-4, BMP-5, BMP-6 and/or BMP-7 in any combination), interleukin-8 (IL-8), interleukin-10 (IL-10), insulin-like growth factor binding protein (IGFBP) (e.g., IGFBP-3; IGFBP-5), a growth factor, a small molecule (e.g., less than about 1000 Da), a regenerative agent and any combination thereof.

In certain embodiments, the scaffold of this invention can comprise layers wherein the layers comprise the following materials and first bioactive agents and second bioactive agents arranged in the following order from exterior to interior: a) a surface layer comprising one or more than one first bioactive agent, a positively charged polyelectrolyte and a negatively charged polyelectrolyte; b) a cross-linked protein; c) one or more than one second bioactive agent and a positively or negatively charged polyelectrolyte; d) a negatively or positively charged polyelectrolyte that has the opposite electrostatic charge of the polyelectrolyte of (c); and e) one or more scaffold component comprising degradable synthetic polymers, degradable natural polymers and any combination thereof.

In a particular embodiment, the scaffold of this invention can comprise: a) an antimicrobial agent and/or SB203580 and/or PD98059 as first bioactive agents, PAH as the positively charged polyelectrolye and PSS as the negatively charged polyelectrolyte; b) collagen crosslinked with genipin; c) PDGF and/or BMP2 as second bioactive agents and polycation poly(allylanion hydrochloride) (PAH) as the positively charged polyelectrolyte; d) polyanion (polyacrylic acid) (PAA) as the negatively charged polyelectrolyte; and e) 50:50 poly(lactic-co-glycolic acid) (PLGA):collagen as the scaffold component.

In some embodiment, the scaffold of this invention can have a symmetrical organization, wherein the layers comprise the following materials and the one or more than one first bioactive agent and one or more than one second bioactive agent arranged in the following order in cross section: a) a surface comprising one or more than one first bioactive agent, a positively charged polyelectrolyte and a negatively charged polyelectrolyte; b) a cross-linked protein; c) one or more than one second bioactive agent and a positively or negatively charged polyelectrolyte; d) a negatively or positively charged polyelectrolyte that has the opposite electrostatic charge of the polyelectrolyte of (c); e) a degradable polymer; f) a negatively or positively charged polyelectrolye; g) one or more than one second bioactive agent that can be the same or different from the one or more than one second bioactive agent of (c) and a positively or negatively charged polyelectrolyte that has the opposite electrostatic charge of the polyelectrolyte of (f); h) cross-linked protein; and i) a surface coating comprising one or more than one first bioactive agent that can be the same or different from the one or more than one first bioactive agent of (a), a positively charged polyelectrolyte and a negatively charged polyelectrolyte.

In particular embodiments, a scaffold of this invention can comprise, in the following order in cross section: a) an antimicrobial agent and/or SB203580 and/or PD98059 as first bioactive agents, PAH as the positively charged polyelectrolye and PSS as the negatively charged polyelectrolyte; b) collagen crosslinked with genipin; c) PDGF and/or BMP2 as second bioactive agents and polycation poly(allylanion hydrochloride) (PAH) as the positively charged polyelectrolyte; d) polyanion (polyacrylic acid) (PAA) as the negatively charged polyelectrolyte; e) 50:50 poly(lactic-co-glycolic acid) (PLGA):collagen as the degradable polymer; f) PAA as the negatively charged polyelectrolyte; g) PDGF and/or BMP2 as second bioactive agents and PAH as the positively charged polyelectrolyte; h) collagen crosslinked with genipin; and i) an antimicrobial agent and/or SB203580 and/or PD98059 as first bioactive agents, PAH as the positively charged polyelectrolye and PSS as the negatively charged polyelectrolyte.

Additionally provided herein is a biocompatible, biodegradable, three-dimensional scaffold having a surface and an interior, said scaffold comprising: a) a multiplicity of layers, wherein the layers comprise materials that degrade at different rates, with layers at the surface of the scaffold degrading prior to layers at the interior of the scaffold; b) one or more than one anti-inflammatory agent located at the surface of the scaffold; and c) one or more than one regenerative agent located at the interior of the scaffold, wherein when the scaffold is exposed to an environment surrounding the scaffold, the anti-inflammatory agent is released into the environment prior to release of the regenerative agent and following release of the anti-inflammatory agent and degradation of the layers at the surface of the scaffold, the regenerative agent is released into the environment, thereby sequentially and separately releasing the anti-inflammatory agent and the regenerative agent into the environment.

The scaffold described above can further comprise an antimicrobial agent located at the surface of the scaffold. In embodiments of such a scaffold, the antimicrobial agent is located in one or more than one outer layer at the surface of the scaffold and the anti-inflammatory agent is located in one or more than one inner layer at the surface of the scaffold, whereby when the scaffold is exposed to the environment surrounding the scaffold, the antimicrobial agent is released into the environment prior to release of the anti-inflammatory agent.

In the scaffold of this invention, the layers can comprise a material selected from the group consisting of: collagen, gelatin, polycation poly(allylanion hydrochloride) (PAH), polyanion (polyacrylic acid) (PAA), polycation poly(styrene sulfonate) (PSS), poly(lactic-co-glycolic acid) (PLGA), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatide, calcium phosphate and any combination thereof.

The present invention further provides a method of sequentially and separately delivering an anti-inflammatory agent and then a regenerative agent to a subject having a disorder in which reduction of inflammation followed by tissue regeneration at a lesion site in the subject is indicated, comprising contacting the lesion site of the subject with the scaffold of claim 1, wherein the one or more than one first bioactive agent comprises an anti-inflammatory agent and the one or more than one second bioactive agent comprises a regenerative agent, for a period of time sufficient to deliver the anti-inflammatory agent to reduce inflammation at the lesion site and then deliver the regenerative agent to regenerate tissue at the lesion site after inflammation has been reduced.

The above-described method of this invention can further comprise sequentially and separately delivering an antimicrobial agent to the subject, comprising contacting the lesion site of the subject with the scaffold, wherein the scaffold comprises one or more than one outer layer and one or more than one inner layer at the surface of the scaffold and wherein the one or more than one first bioactive agent further comprises an anti-microbial agent located in the one or more than one outer layer and the anti-inflammatory agent is located in the one or more than one inner layer, for a period of time sufficient to deliver the antimicrobial agent to treat infection at the lesion site and then deliver the anti-inflammatory agent to reduce inflammation at the lesion site and then deliver the regenerative agent to regenerate tissue at the lesion site after inflammation has been reduced.

In the methods of this invention, the disorder can be diabetic ulcer and/or periodontal disease.

In certain embodiments of the methods of this invention, the lesion site is contacted with the scaffold for a period of time sufficient to reduce inflammation by more than 50%.

Additionally provided herein is a method of treating diabetic ulcer in a subject, comprising contacting the diabetic ulcer of the subject with an effective amount of the scaffold of this invention, as well as a method of treating periodontal disease in a subject, comprising contacting diseased periodontal tissue of the subject with an effective amount of the scaffold of this invention and a method of treating a lesion site and/or wound site and/or surgical site in a subject, comprising contacting the lesion site and/or wound site and/or surgical site with an effective amount of the scaffold of this invention.

Further provided herein is a method of enhancing tissue regeneration and/or healing at a lesion site and/or wound site and/or a surgical site in a subject by first treating or controlling infection and/or reducing inflammation at the site, thereby enhancing tissue regeneration and/or healing at the site, comprising contacting the lesion site and/or wound site and/or surgical site with an effective amount of the scaffold of this invention.

In the methods of this invention, the amount of inflammation can be substantially reduced, which means a reduction of inflammation at the lesion site and/or wound site and/or surgical site by at least 40%, 50%, 60%, 70%, 80%, 90% or 100%. The percent reduction in inflammation can be determined by comparison with a control (e.g., by comparison with a nontreated lesion site, wound site and/or surgical site and/or by determining an amount of inflammation prior to treatment according to methods known in the art and measuring the amount of reduction in inflammation upon treatment as described herein.

### BRIEF DESCRIPTION OF THE FIGURES

FIG. 1. Overview of periodontal inflammation and intracellular signaling. Periodontal bone loss occurs through activation of the immune response by plaque-associated constituents, e.g., LPS. This activation occurs in the periodontal tissues by a wide variety of cells including gingival fibroblasts, macrophages and osteoblasts/stromal cells. Cytokines generated directly or indirectly activate osteoclastogenesis, resulting in periodontal tissue loss. Insert indicates that multiple MAPK signaling pathways are activated in response to LPS and generate cytokines and MMPs that contribute to tissue and bone destruction within the periodontium.

FIGS. 2A-D. A. E. coli and A. actinomycetemcomitans LPS activate MAPK pathways in macrophages. RAW 264.7 cells were stimulated for 15 min with LPS and whole cell lysates were used for Western blot analysis. The p38 MAPK inhibitor, SB203580 (10 μg/ml) was added to indicated cultures 10 min prior to LPS stimulation. Results indicate that p38 and MK2, a downstream p38 substrate, are markedly activated by A. actinomycetemcomitans LPS. B. A. actinomycetemcomitans LPS stimulation of IL-6 requires p38 MAPK signaling. Wild-type and p38 MAPK deficient cells were stimulated with A. actinomycetemcomitans LPS for 24 hrs (white bars). Cells were rescued with pcDNA containing p38a cDNA (p38α) in p38^{-/-} cells but the dominant negative mutant (p38AF) was not able to restore LPS-induced IL-6 expression. C. The inhibitory effect of PD98059 (ERK inhibitor) on LPS-stimulated MMP-1 expression and AP-1 DNA-binding activity. U937 macrophages were treated with 100 ng/ml of LPS in the presence or absence of 10 μM of PD98059 for 24 h. After the treatment, MMP-1 in culture medium was quantified by ELISA. D. Nuclear proteins were extracted from cells for electrophoretic mobility shift assay to determine AP-1 DNA-binding activity.

FIGS. 3A-D. Signaling pathways activated in the LPS-induced model of experimental periodontal disease in rats. Representative images of signaling pathways activated (A) and the histological aspect in hematoxylin/eosin (H/E)-stained sections (B) of the gingival tissue of rats according to the period (5, 15 or 30 days) since beginning LPS injections (3 times/week). Densitometric analysis of Western blot results (C) and stereometric analysis indicate that p38 and ERK MAP kinases are significantly activated throughout the 30-day period of observation and this activation parallels the severity of inflammation in the gingival tissues, as indicated by the stereometric analysis results (D), demonstrating significant increases in inflammatory cells and decrease in fibroblastic cell density. There is also a noticeable trend towards a decrease in collagen content and an increase in the vascularization of the tissues (n=5).

FIGS. 4A-D. A. μCT isoform displays from 8-week A. actinomycetemcomitans LPS-injected rat maxillae exhibit dramatic palatal and interproximal bone loss. B. Cementoenamel junction (CEJ) to the alveolar bone crest (ABC) was used to determine alveolar bone loss. Linear bone loss as measured from the CEJ to ABC (Mean±SEM). Significant bone loss (p<0.01) was observed between control (n=6) and A. actinomycetemcomitans LPS injected rats (n=12). Significant reduction of LPS-induced periodontal bone loss was observed in SD-282 treated rats (**p<0.01 for SD-282 [15 mg/kg; n=8] and *p<0.05 for SD-282 [45 mg/kg; n=8]). C. Bone area fraction (BAF; Mean±SEM) in SD-282 treated rats with LPS-induced periodontal bone loss indicates a significant protective effect is observed relative to interproximal area bone loss (*p<0.01 for both SD-282 [15 mg/kg and 45 mg/kg]). D. Data are presented as bone volume fraction (BVF) (mean±SEM). Significant bone loss (***p<0.001) was observed between control (n=6) and A. actinomycetemcomitans LPS injected rats (n=12). In SD-282 treated rats, significant protection of LPS-induced periodontal bone loss was observed in both treatment groups (*p<0.05 and **p<0.01; n=8 per group).

FIGS. 5A-C. Differential activation of signaling pathways in MKP-1^{+/+} and MKP-1^{-/-} cells. (A) Primary BMSCs were stimulated for 10, 30, 60, 120 and 240 minutes with LPS from A. actinomycetemcomitans. Besides sustained activation of p38 MAPK, constitutive and prolonged activation of NF-kB can be observed. Also, increased activation of JNK MAP kinase is observed, as well as increased activation of MKP-5, a p38 MAPK substrate. These results suggest a compensatory mechanism between MKP-1 and MKP-5 and a ‘shift’ in signal transduction between p38 and JNK MAP kinases. MKP-1 reduces inflammatory-induced bone loss in experimental periodontitis. (B) Mouse maxillary images from μCT. A.a. LPS (20 μg) was injected directly into the left palatal region between the 1^{st} and 2^{nd} molar. PBS was used as control in the right side. The injections were performed 3 times a week for 4 weeks. (C) The bone volume fraction (BVF) was analyzed by MICROVIEW software (GE Healthcare). Statistical analysis was performed by Student's t test.

FIGS. 6A-C. Human periodontal disease tissue has higher levels of activated p38 and ERK MAPK. Human biopsy tissue was obtained during periodontal surgery. Clinical periodontal parameters were obtained along with BANA analysis. Immunohistochemistry was performed on fixed tissues for (A) P-p38, (B) P-ERK, and (C) P-JNK MAPK and blindly scored by a board certified oral pathologist. Intensity scoring (0-3 scale) is presented here. Both P-p38 and P-ERK showed a trend towards increased scoring with clinical inflammation (as measured by Gingival Index (Loe and Silness)). P-p38 reached significance (p=0.015). P-JNK did not correlate with clinical inflammation.

FIGS. 7A-H. Biodegradable nanofibrous scaffolds of both synthetic and natural materials fabricated using electrospinning technology (upper panel). (A) PLGA nanofibrous porous scaffold, (B) Collagen nanofibrous scaffold, (C) Polycaprolactone (PCL)-collagen hybrid scaffold, and (D) PLGA-collagen hybrid scaffold. Biodegradable nanofibrous scaffolds of different patterns fabricated using electrospinning (lower panel). (E-F) Wavy PCL nanofibers (DiO labeled) embedded in linear elastic polyurethane nanofibers (DiI labeled) to mimic the wavy collagen fibers and elastin fibers in the natural blood vessel wall. (G) Unidirectional aligned PCL nanofibrous scaffolds (DiI labeled). (H) bidirectional aligned PCL nanofibrous scaffolds (DiI labeled).

FIGS. 8A-D. PLGA-collagen scaffolds can retain small molecules and growth factors. The morphology of PLGA-collagen nanofibers before nanoLbL (A) and after nanoLbL coating (B). The SC203850 and BMP-2 daily release (C) and PD98059 daily release (D) from one 15 mm diameter and 1 mm thick scaffold.

FIGS. 9A-B. Human U937 macrophages were exposed to control scaffold, PD98059-embedded scaffold, or SB203580-embedded scaffold in the presence or absence of LPS (100 ng/ml) for 24 hrs. After the exposure, culture medium was collected for ELISA to quantify IL-6 (A) or MMP-1 (B).

FIG. 10. BMP-2 induces ectopic bone formation. Micro-CT of BMP-2 loaded scaffold after four weeks implantation subcutaneously into the back of rats. One slice in cross section (A) and one slice in horizontal section (B).

FIG. 11. NR8383 monocytes respond to LPS in a MAPK-dependent manner. Rat monocyte/macrophage cells were transduced with adenovirus Ad5-MKP-1 or control virus (Ad5.bgal) and then stimulated with LPS for indicated time points. Data indicate that overexpression of MKP-1 can significantly block LPS-induced IL-6 expression. Since MKP-1 can dephosphorylate p38, JNK and ERK MAP kinases, these data support other data that indicate that MAPK is critical for cytokine expression.

FIG. 12. Schematic of the nanoLbL functionalization of scaffolds. Anti-inflammatory agents, such as p38 and/or ERK inhibitors will be coated on the top or outer surface and growth factors will be embedded on the bottom or inner compartment of the nanoLbL coating.

### DETAILED DESCRIPTION

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a biomolecule or agent of this invention, dose, time, temperature, and the like, is meant to encompass variations of ±20%, ±10%, ±5%, ±1%, ±0.5%, or even ±0.1% of the specified amount.

In one aspect, the present invention provides a new strategy for treatment of an inflamed lesion site, wound site, surgical site and/or disease site in need of tissue regeneration by sequentially and separately delivering one or more than one antimicrobial agent and/or one or more anti-inflammatory agent to the site to first treat or control infection and/or reduce inflammation and then delivering one or more regenerative agents to the site to promote and/or enhance tissue regeneration and/or healing. Specifically, in one embodiment, the present invention provides a biocompatible, biodegradable, three-dimensional scaffold comprising, consisting essentially of and/or consisting of: a) a multiplicity of layers, wherein the layers comprise materials (e.g., nanofibers) that degrade at different rates, with layers at the surface of the scaffold degrading prior to layers at the interior of the scaffold; b) one or more than one first bioactive agent located at the surface of the scaffold; and c) one or more than one second bioactive agent located at the interior of the scaffold, wherein when the scaffold is exposed to an environment surrounding the scaffold, the first bioactive agent is released into the environment prior to release of the second bioactive agent and following release of the first bioactive agent and degradation of the layers at the surface of the scaffold, the second bioactive agent is released into the environment, thereby sequentially and separately releasing the first and second bioactive agents into the environment.

In the scaffold described above, the first bioactive agent can be, but is not limited to, an antimicrobial agent, an inhibitor of interleukin-1 (IL-1), an inhibitor of interleukin-6 (IL-6), an inhibitor of tumor necrosis factor alpha (TNF-α), an inhibitor of matrix metalloproteinase (MMP) 1, 2, 8 and/or 9, an inhibitor of p38 mitogen activated protein kinase (MAPK), an inhibitor of extracellular signal-related kinase (ERK) (e.g., ERK1; ERK2), SBR203580 (p38 inhibitor), PD98059 (ERK inhibitor), U0126 (inhibitor of MMP expression) simvastatin (inhibitor of MMP-1 expression), any anti-inflammatory agent now known or later identified and any combination thereof.

Furthermore, in the scaffold described above, the second bioactive agent can be, but is not limited to, hepatocyte growth factor (HGF; e.g., HGF-1), stromal cell-derived factor (SDF-1), transforming growth factor beta (TGF-β; e.g., TGF-β1, TGF-β3), platelet derived growth factor (PDGF) (e.g., BECAPLERMIN; REGRANEX®, PDGF-BB), platelet releasate, epidermal growth factor (EGF), fibroblast growth factor (FGF; e.g., FGF-2), granulocyte macrophage colony stimulating factor (GM-CSF), keratinocyte growth factor-2 (KGF-2), insulin-like growth factor (IGF; e.g., IGF-I, IGF-II), bone morphogenetic protein (BMP; e.g., BMP-2, BMP-4, BMP-5, BMP-6 and/or BMP-7 in any combination), interleukin-8 (IL-8), interleukin-10 (IL-10), insulin-like growth factor binding protein (IGFBP; e.g., IGFBP-3; IGFBP-5), a growth factor, a small molecule (e.g., less than about 1000 Da), and any combination thereof.

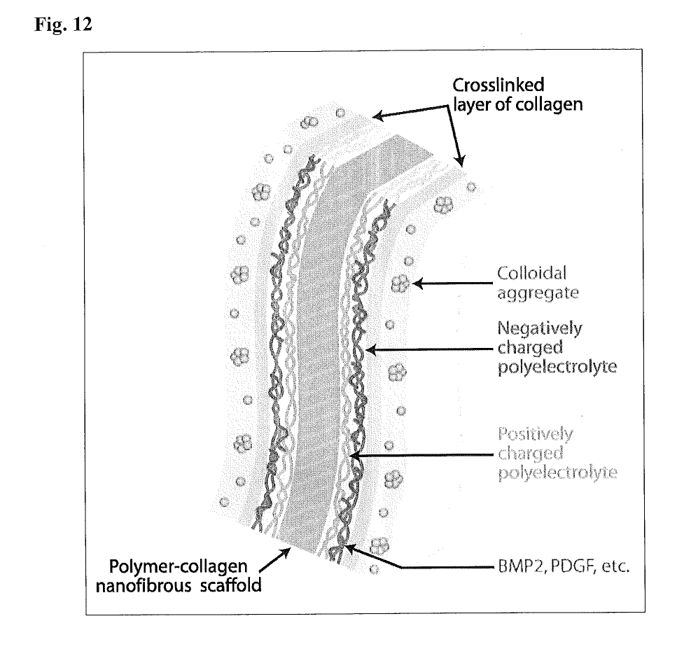

A nonlimiting example of a scaffold of this invention, showing various elements that can be included in the scaffold of this invention and their orientation and organization in the exemplary scaffold, is provided herewith as FIG. 12.

In certain embodiments of this invention the scaffold can comprise, consist essentially of and/or consist of the following elements arranged in the following order from top (e.g., an upper or exterior surface) to bottom (e.g., a lower or interior surface): a) a surface layer (e.g., a colloidal aggregate) comprising one or more than one first bioactive agent, a positively charged polyelectrolyte and a negatively charged polyelectrolyte; b) cross-linked collagen (e.g., to function as a barrier layer to prevent the release of inner layers); c) one or more than one second bioactive agent and a positively or negatively charged polyelectrolyte; d) a positively or negatively charged polyelectrolyte that has the opposite electrostatic charge of the polyelectrolyte of (c); and e) degradable polymer.

In some embodiments, an outer layer (e.g., at an upper or exterior surface) at the surface of the scaffold of this invention can comprise, consist essentially of or consist of one or more antibiotics and/or antimicrobial agent (e.g., antimicrobial peptide), and an inner layer (e.g., inside the outer layer) at the surface of the scaffold can comprise, consist essentially of or consist of one or more anti-inflammatory agents and an interior layer (e.g., inside the inner layer) can comprise, consist essentially of or consist of one or more regenerative factors (e.g., growth factors). These can be present in any order relative to one another and/or relative to other components of the scaffold. Furthermore, there can be multiple (e.g., 2 or more, including 3, 4, 5, 6, 7, 8, 9, 10, etc.) outer layers, inner layers and/or interior layers. As a nonlimiting example, the scaffold of this invention can have three outer layers of one or more than antimicrobial agent, four inner layers of one or more than one anti-inflammatory agent and six interior layers of one or more regenerative factor (e.g., growth factor). Increasing the number of nano-layers allows for longer release time of each agent within the layers.

In embodiments in which the scaffold is organized or oriented such that it does not have a top and bottom, but rather has an exterior and interior (e.g., an exterior surface that is present on all sides of the scaffold and an interior region that is not exposed at the outside of the scaffold, such as in a spherical or symmetrical orientation), the scaffold can comprise, consist essentially of and/or consist of the following elements arranged in the following order in cross section (e.g., in cross section from a first exterior position, through the interior, to a second exterior position opposite the first exterior position): a) a surface layer (e.g., a colloidal aggregate) comprising one or more than one first bioactive agent, a positively charged polyelectrolyte and a negatively charged polyelectrolyte; b) a cross-linked protein (e.g., collagen, as a barrier layer); c) one or more than one second bioactive agent and a positively or negatively charged polyelectrolyte; d) a negatively or positively charged polyelectrolyte that has the opposite electrostatic charge of the polyelectrolyte of (c); e) a degradable polymer; f) a negatively or positively charged polyelectrolye; g) one or more than one second bioactive agent that can be the same or different from the one or more than one second bioactive agent of (c) and a positively or negatively charged polyelectrolyte that has the opposite electrostatic charge of the polyelectrolyte of (f); h) a scaffold component; and i) a surface layer (e.g., colloidal aggregate) comprising one or more than one first bioactive agent that can be the same or different from the first bioactive agent of (a), a positively charged polyelectrolyte and a negatively charged polyelectrolyte.

In particular embodiments, in the scaffolds of this invention described above having elements (a)-(e) or (a)-(i), these elements can comprise, consist essentially of and/or consist of the following: a) an antimicrobial agent and/or SB203580 and/or PD98059 as first bioactive agents, polycation poly(allylanion hydrochloride) (PAH) as the positively charged polyelectrolyte and polycation poly(styrene sulfonate) (PSS) as the negatively charged polyelectrolyte; b) collagen crosslinked with genipin; c) PDGF and/or BMP-2 as second bioactive agents and PAH as the positively charged polyelectrolyte; d) polyanion (polyacrylic acid) (PAA) as the negatively charged polyelectrolyte; e) a natural polymer (such as collagen, gelatin, chitosan, laminin, etc., including any combination thereof) and/or a degradable polymer (e.g., PLA, PGS, PLA, PLGA, polycaprolactone, polyurethane, etc., including any combination thereof) as a scaffold component; f) PAA as the negatively charged polyelectrolyte; g) PDGF and/or BMP-2 (and/or any growth factor) as second bioactive agents and PAH as the positively charged polyelectrolyte; h) collagen crosslinked with genipin or other crosslinking agents; and i) an antimicrobial agent and/or SB203580 and/or PD98059 and/or other inhibitors and/or anti-inflammatory agents as first bioactive agents, PAH as the positively charged polyelectrolye and PSS as the negatively charged polyelectrolyte.

In further embodiments, the present invention provides a biocompatible, biodegradable, three-dimensional scaffold comprising, consisting essentially of and/or consisting of: a) a multiplicity of layers, wherein the layers comprise materials that degrade at different rates, with layers at the surface of the scaffold degrading prior to layers at the interior of the scaffold; b) one or more than one antimicrobial agent and/or one or more than one anti-inflammatory agent located at the surface of the scaffold; and c) one or, more than one regenerative agent located at the interior of the scaffold, wherein when the scaffold is exposed to an environment surrounding the scaffold, the antimicrobial agent and/or the anti-inflammatory agent is released into the environment prior to release of the regenerative agent and following release of the antimicrobial agent and/or anti-inflammatory agent and degradation of the layers at the surface of the scaffold, the regenerative agent is released into the environment, thereby sequentially and separately releasing the antimicrobial agent and/or anti-inflammatory agent and the regenerative agent into the environment over time.

In the scaffolds of this invention, the layers (e.g., scaffold components) can comprise, consist essentially of and/or consist of a material that can be, but is not limited to, collagen (e.g., collagen I, IV), polycation poly(allylanion hydrochloride) (PAH), polyanion (polyacrylic acid) (PAA), polycation poly(styrene sulfonate) (PSS), poly(lactic-co-glycolic acid) (PLGA), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate (e.g., poly-3-hydroxybutyrate), polylactic acid, polydioxanone, chitosan, laminin, glycosaminoglycan (e.g., hyaluronic acid), proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, gelatin and any combination thereof.

Gelatin is a polyampholyte naturally derived from denatured collagen. Like many other proteins, it has a heterogeneous charge distribution on the surface with the presence of both negatively charged and positively charged patches. The peptide sequence of gelatin facilitates cell attachment and proliferation. Gelatin scaffolds have been shown to promote chondrogenic differentiation in bone marrow stem cells (BMSC) and adipose-derived mesenchymal stem cells (MSC). Adding gelatin to a composite scaffold has been shown to increase type II collagen expression by BMSC in vitro.

In some embodiments, the scaffold of this invention can be treated with a crosslinking and/or catalyzing agent [e.g., 1-ethyl-(3-3-dimethylaminopropyl carbodiimide hydrochloride (EDC); N,N′-dicyclohexylcarbodiimide (DCC); N,N′-diisoproplycarbodiimide (DIC), genipin and any other crosslinking and/or catalyzing agent known in the art for crosslinking proteins, in any combination]. In certain embodiments of the methods of this invention, the nanofibers of the scaffold are crosslinked with genipin.

As used herein, the term “nanofiber” means a fiber having at least one dimension of 100 nm or less.

As used herein, the term “polyelectrolyte” refers to a polymer having repeating units that bear an electrolyte group, imparting either a positive charge (e.g., a “positively charged polyelectrolyte”) or a negative charge (e.g., a “negatively charged polyelectrolyte”) to the polymer.

Also as used herein, the terms “degrade, degradation, degrading” and derivatives thereof mean that a material or composition decomposes into smaller and/or less complex molecules or atoms; that a material or composition is diminished, becomes reduced in complexity and/or is broken down; and/or that a material or composition dissolves, dissipates, erodes and or is released into a surrounding environment.

Furthermore, the terms interior or inner or inside when described with reference to the scaffold of this invention mean an area or region or location that has no direct contact with a surrounding environment prior to degradation of the scaffold.

The terms exterior, upper, outer, top, surface or outside when described with reference to the scaffold of this invention mean an area or region or location that is in direct contact with a surrounding environment prior to and/or at the initiation of degradation of the scaffold.

As used herein, the term “environment” describes the physical location or position of the scaffold. For example, an environment of this invention can be the interior, exterior, surrounding area and/or surface of a body cavity and/or lesion and/or wound site of a subject. Upon placement, delivery, introduction and/or deposit of the scaffold of this invention into such an environment, release of first bioactive agents on the surface or exterior or top or outside of the scaffold is initiated, simultaneously with and/or followed by degradation of layers of the scaffold, resulting in release of second bioactive agents into the environment.

As used herein, the term “antimicrobial agent” means any agent that kills, inhibits the growth of, or prevents the growth of a bacterium (including mycoplasma), fungus, yeast, or virus. Suitable antimicrobial agents of this invention include, but are not limited to, antibiotics such as vancomycin, bleomycin, pentostatin, mitoxantrone, mitomycin, dactinomycin, plicamycin and amikacin. Other antimicrobial agents include, but are not limited to, antibacterial agents such as 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, amoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefmenoxime, cefininox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clindamycin phosphate, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin and vancomycin. Exemplary antimicrobial agents may also include, but are not limited to, anti-fungals, such as amphotericin B, azaserine, candicidin(s), chlorphenesin, dermostatin(s), filipin, fungichromin, mepartricin, nystatin, oligomycin(s), perimycin A, tubercidin, imidazoles, triazoles, and griesofulvin. Further exemplary antimicrobial agents can include, but are not limited to anti-virals, such as acyclovir, valacyclovir, famcyclovir, gancyclovir, amantadine and others known in the art.

An antimicrobial agent of this invention can also be an antimicrobial peptide, including a plant derived antimicrobial peptide and an animal antimicrobial peptide. Antimicrobial peptides (AMPs) are short sequence peptides with generally fewer than 50 amino acid residues, which have antimicrobial activity against microorganisms. They are a first line of defense in plants and animals which are ubiquitous in nature with high selectivity against target organisms, and resistance against them is much less observed compared with current antibiotics (Zasloff, 2002).

AMPs are diverse and can be subdivided into two major groups based on their electrostatic charges, which are the most important characteristic of AMPs (Vizioli and Salzet, 2002). The largest group of AMPs is that of cationic molecules, which are wildly distributed in plants and animals. The much smaller group of AMPs is that of non-cationic molecules including anionic peptides, aromatic peptides and peptides derived from oxygen-binding proteins. Compared with the first group, the non-cationic peptides are scarce and often the term “antimicrobial peptides (AMPs)” is used to refer only to cationic AMPs (Zasloff, 2002; Keymanesh, 2009).

On the basis of structural features, cationic AMPs can be subdivided into three classes: (1) linear peptides often adopting α-helical structures; (2) cysteine-rich open-ended peptides containing a single or several disulfide bridges; and (3) cyclopeptides forming a peptide ring (Montesinos, 2007). However, they also share certain common structural characteristics such as (1) amino acid composition in which cationic and hydrophobic residues are most abundant; (2) amphipathicity; and (3) a remarkable diversity of structures and conformations even including some non-conventional and extended structures (Vizioli and Salzet, 2002; Keymanesh, 2009). In fact, the second characteristic, amphipathicity, in many cases is membrane-induced, and this is an important property of cationic AMPs which can facilitate their interactions with microbial membranes (Zasloff, 2002). Some cationic AMPs are enriched in certain amino acids. For example, many cationic AMPs are rich in cysteines forming a single or several disulfide bridges (e.g., Ib-AMP4 from balsamine and penaeidins from shrimp), which makes their structures more compact and stable under various biochemical conditions such as protease degradation and so on. This group of AMPs is widespread in nature, including plants, animals, insects, and fungi, and exhibit a significant sequence and structure diversity (Vizioli and Salzet, 2002).

The present invention additionally provides methods of using the scaffolds of this invention. In one embodiment, the present invention provides a method of sequentially and separately delivering an antimicrobial agent and/or an anti-inflammatory agent first and then delivering a regenerative agent to a subject having a disorder in which treatment of infection and/or reduction of inflammation followed by tissue regeneration at a lesion and/or wound site in the subject is indicated and/or desired, comprising contacting the lesion and/or wound site of the subject with the scaffold of this invention for a period of time sufficient to first deliver the antimicrobial agent to treat infection and/or the anti-inflammatory agent to reduce inflammation at the lesion and/or wound site and then deliver the regenerative agent to regenerate tissue at the lesion and/or wound site after inflammation has been reduced.

In methods of this invention, the disorder can be any disorder in which treatment of infection and/or reduction of inflammation followed by tissue regeneration at a lesion site and/or wound site and/or disease site and/or surgical site in a subject is indicated and/or desired. In some embodiments of this invention, the disorder can be, but is not limited to, diabetic ulcer, periodontal disease, chronic lesions, wounds, and any combination thereof.

In the methods of this invention in which inflammation is reduced, the inflammation can be reduced (e.g., reduced by 10%, 20%, 30%, 40%, 50%, 60%, 70%, 80%, 90%, 95% or 100%) as compared to the amount of inflammation present at a lesion and/or wound site prior to contact with a scaffold of this invention. In particular embodiments, the amount of inflammation can be substantially reduced (e.g., by at least about 30%, 40%, 50%, 60%, 70%, 80%, 90%, 100%). Thus in some embodiments of this invention, the lesion and/or wound site is contacted with the scaffold for a period of time sufficient to reduce inflammation by at least about 50% (i.e., to substantially reduce inflammation).

The amount of inflammation can be determined by measuring the amount of pro-inflammatory agents (e.g., IL-6; MMP-1, etc.) present at the lesion and/or wound site according to protocols as described herein and as are well known in the art. A reduction in the amount of inflammation is also determined by measuring the amount of pro-inflammatory agents at the lesion and/or wound site before and after contact with a scaffold of this invention. A reduction in the amount of the pro-inflammatory agents under analysis indicates a reduction in inflammation and the amount of inflammation reduction as measured by percent can be determined from such assays according to methods standard in the art.

In particular embodiments of this invention, a method is provided of treating diabetic ulcer in a subject, comprising contacting the diabetic ulcer of the subject with an effective amount of the scaffold of this invention.

In other embodiments, a method is provided of treating periodontal disease in a subject, comprising contacting diseased periodontal tissue of the subject with an effective amount of the scaffold of this invention.

In further embodiments, a method is provided of healing a lesion and/or wound in a subject, comprising contacting the lesion and/or wound of the subject with an effective amount of the scaffold of this invention.

In addition, the present invention provides a method of enhancing tissue regeneration and/or healing at a lesion and/or wound site in a subject by first reducing inflammation at the lesion and/or wound site, thereby enhancing tissue regeneration and/or healing at the lesion and/or wound site, comprising contacting the lesion and/or wound site with an effective amount of the scaffold of this invention. In some embodiments, the inflammation is substantially reduced (e.g., by at least about 50%).

As described herein, the scaffold of this invention can be contacted at a site where tissue regeneration is needed and/or desired such that one or more regenerative agents is released at the site after anti-inflammatory agents have been released at the site, resulting in a reduction or elimination of inflammation at the site. The regenerative agents of this invention are agents (e.g., biomolecules) that promote tissue regeneration (e.g., via recruitment and/or activation of endogenous stem cells to the site of regeneration).

In some embodiments, such regenerative agents or biomolecules can be delivered sequentially to cue endogenous stem cells for mobilization and migration, proliferation and/or chondrogenesis. In certain embodiments, endogenous stem cells can be recruited into the scaffold first, which then proliferate and differentiate into the desired cell type. In a particular example, in which cartilage repair is the desired type of tissue regeneration, endogenous stem cells from synovium membrane and underlying bone can be recruited into the scaffold first, which then proliferate and differentiate into chondrocytes. Thus, the spatio-temporal delivery system of this invention using biocompatible nanoparticles, hydrogels, and scaffolds can mimic the events and/or stages of normal tissue healing and regeneration.

A particular aspect of this invention is the separate and sequential delivery of different agents or biomolecules to a subject via the scaffold of this invention, e.g., at a site where 1) inflammation is present and there is a need or desire to reduce the inflammation, and 2) tissue regeneration is indicated, needed and/or desired. Biomolecule delivery requirements are to be taken into account when selecting materials for scaffold fabrication. Both the method of biomolecule incorporation and the degradation rate of the biomaterial will determine the release kinetics of the biomolecule. Temporal release features to be considered include the ability to deliver or release each of the different biomolecules over a period of time, to delay the onset of delivery, and/or to generate a sustained release.

Furthermore, in some embodiments, short-term biomolecule and/or signal delivery can be achieved by encapsulating the biomolecule(s) in nanospheres and/or microspheres, the production and use of which are well-known in the art. Nanoparticles and microspheres can be delivered to the subject via a scaffold of the present invention or can be delivered directly to the subject. Material selection for the nanoparticle and microsphere diameter will determine the length of the biomolecule delivery period. Additionally, biomolecule delivery corresponding to cell infiltration can be achieved, e.g., by using an enzymatically sensitive hydrogel.

Cueing mesenchymal stem cells (MSC) to mobilize, migrate, proliferate, and/or differentiate is key to engineering a tissue regeneration and/or healing response in tissues, such as, for example, cartilage. Sources of MSCs include bone marrow, periostium and adipose tissue. The synovial membrane has also shown to be a rich source of MSCs. There are many biomolecules, particularly growth factors, which play a role in healing and tissue regeneration. Candidates for engineering the healing cascade include members of the bone morphogenic protein (BMP) family known to regulate cell fate determination and promote chondrogenesis and osteogenesis. BMPs with potential for cartilage regeneration include BMP-2, BMP-4, BMP-5, BMP-6, and BMP-7. BMP-4 induces chondrogenic maturation of MSC, suppresses hypertrophy and stimulates type II collagen and aggrecan production. BMP-7 upregulates chondrocyte metabolism and protein synthesis. Culturing of MSCs with bFGF promotes maintenance of multipotency and chemotaxis. Hepatocyte growth factor and stromal cell-derived factor-1 have both been reported to have a strong chemotaxic effect on MSC. Platelet derived growth factor is a mitogenic and chemotactic factor for cells of mesenchymal origin. Transforming growth factor β-1 and β-3 are known to induce and maintain the chondrogenic phenotype. Production of extracellular matrix (ECM) is promoted and hypertrophy is inhibited. Insulin-like growth factor-I and -II stimulate directed migration in bone-marrow-derived MSC. Insulin-like growth factor I also stimulates proteoglycan production in a dose-dependent manner. Interleukin 10 has immunosuppression activity and may inhibit the migration of macrophages to the defect site. MSC migrate when stimulated with interleukin 8. Regenerative agents and biomolecules of the present invention can be present as a protein or biologically active peptide thereof or in the form of a nucleic acid encoding the protein or biologically active peptide thereof.

Accordingly, in some embodiments, the scaffold of the present invention can be used for temporally controlled biomolecule delivery to a subject of this invention. In further embodiments, the biomolecules in the form of proteins, peptides and/or nucleic acids can be delivered directly to the subject. Biomolecules in the form of proteins, peptides and/or nucleic acids can be incorporated into the scaffold at any step in the fabrication of the scaffold. Thus, the biomolecule can be incorporated at a pre-fabrication step, during fabrication or post-fabrication. Therefore, biomolecules can be attached to a separate component of a scaffold prior to fabrication and/or biomolecules can be attached to and/or immobilized on the surface of the scaffold and/or incorporated into the scaffold prior to and/or after curing. In some embodiments of the invention, at least one biomolecule is bound directly (i.e., without any linking or intervening material) to the scaffold. Biomolecules can be attached directly to the scaffold via, for example, physical electrostatic force, wherein the negative charges in the biomolecule(s) bind with the positive charges in the scaffold materials. Biomolecules can also be attached directly to the scaffold via chemically covalent binding (e.g., by EDC chemistry). A further example of direct binding of biomolecules to the scaffold is via chemical crosslinking such as photocrosslinking. Biomolecules with photocurable groups can be co-cross-linked with photocurable materials of the scaffold.

In other embodiments, at least one biomolecule can be bound to the scaffold through a linking molecule (i.e., a molecule attached at one site to the biomolecule and attached at a different site to the scaffold). Linking molecules of the invention include, but are not limited to, heparin and heparin sulphate. In particular embodiments of the invention, at least one biomolecule is bound to the scaffold through heparin. In embodiments in which heparin is used as a linking molecule, biomolecules can be used that bind to the heparin by electrostatic force or specific binding. For example, heparin has specific binding with TGF-B1, IL-10, HGF, FGF and others, as is well known in the art. Furthermore, heparin is negatively charged and can bind positively charged biomolecules via electrostatic forces. Additional linking molecules of this invention include heparin analogs and modified polysaccharides, e.g., as described in Frank et al. (J. Biol. Chem. 278 (44):43229-43235 (2003)).

In some embodiments, the biomolecules of this invention can be attached to the scaffold directly and/or via a linking molecule in any proportion and/or combination. For example, the same biomolecule can be attached to the scaffold both directly and via a linking molecule and/or multiple biomolecules can be attached to the scaffold in a configuration such that some biomolecules are attached directly and other biomolecules are attached via a linking molecule. Furthermore, more than one linking molecule can be used in the same scaffold, in any combination. Thus, the present invention further comprises embodiments wherein some biomolecules are bound directly to the scaffold and some biomolecules are bound to the scaffold via a linking molecule. The biomolecules attached to the scaffold directly and/or via a linking molecule can be the same biomolecule or different biomolecules in any combination and in any ratio or percentage relative to one another.

In certain embodiments, the scaffold can be constructed so that its placement or positioning in the environment where inflammation reduction and tissue regeneration is to occur is such that it allows for different regenerative agents to be released in different locations to initiate regeneration of different tissue types. As one nonlimiting example, a scaffold of this invention could have a “right side” and a “left side” in the interior of the scaffold and the right side can comprise, consist essentially of or consist of one or more regenerative agents that promote and/or enhance regeneration of tooth tissue and the left side can comprise, consist essentially of or consist of one or more regenerative agents that promote regeneration of gum tissue. The scaffold can be placed or positioned into a lesion or cavity between gum tissue and a tooth in the mouth of a subject with periodontal disease in an orientation whereby the right side of the scaffold is in proximity to the tooth and the left side of the scaffold is in proximity to the gum tissue. In such an embodiment, after the anti-inflammatory agents on the scaffold have been released into the environment to reduce inflammation, the respective regenerative agents are released into the environment to act directly on the respective tissues proximal to where these agents are released.

As used herein, the term “regenerative agent” or “regenerative biomolecule” describes an agent or molecule that functions to promote, initiate and/or enhance regeneration of tissue, including but not limited to, skin, bone, tooth, muscle, connective tissue, cells, cartilage, tendon, ligament, mucous membrane and any combination thereof. A regenerative agent or biomolecule of the present invention includes, but is not limited to, a differentiation stimulating biomolecule, a chemotaxis stimulating molecule, a proliferation stimulating biomolecule, a mobilization stimulating biomolecule, or any combination thereof.

In some embodiments of the invention, the differentiation stimulating biomolecule includes, but is not limited to, a bone morphogenic protein (BMP, including BMP-1, BMP-2, BMP-4, BMP-5, BMP-6, BMP-7, BMP-8a and/or BMP-9), a transforming growth factor (TGF), including TGF-alpha, TGF-beta 1, TGF-beta 2 and TGF-beta 3, vitamin B12, an insulin-like growth factor-I (e.g., IGF-I; Stem Cells 22:1152-1167 (2004)), IGF-II, or any combination thereof.

In other embodiments, the chemotaxis and/or proliferation stimulating biomolecule includes, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), an interleukin (e.g., interleukin-8; interleukin-10) or any combination thereof.

In further embodiments of the invention, the mobilization stimulating biomolecule includes, but is not limited to, a hepatocyte growth factor (HGF), a stromal cell-derived growth factor-1 (SDF-1), a platelet derived growth factor-bb (PDGF-bb), an insulin-like growth factor (IGF), including IGF-I and IGF-II, an insulin-like growth factor binding protein (IGFBP), including IGFBP-1, IGFBP-2, IGFBP-3, IGFBP-4, IGFBP-5, IGFBP-6, IGFBP-7, TGF-beta 1, TGF-beta 3, BMP 2, BMP 4, BMP 7, basic fibroblast growth factor (bFGF), FGF, EGF, an interleukin (e.g., interleukin-8; interleukin-10) or any combination thereof.

In still further embodiments, the bone morphogenic protein (BMP) includes, but is not limited to, BMP-2, BMP-4, BMP-5, BMP-6, BMP-7, or any combination thereof. In yet other aspects of the invention, the transforming growth factor (TGF) includes, but is not limited to, TGF β-1, TGF β-3, or any combination thereof. In other aspects of the invention, the insulin-like growth factor (IGF) includes, but is not limited to, IGF-I, IGF-II, or any combination thereof. Thus, in particular aspects of the invention, the differentiation stimulating biomolecule that is an insulin-like growth factor is IGF-I. In other aspects of the invention, the chemotaxis and/or proliferation stimulating biomolecule that is an insulin-like growth factor is IGF-I, IGF-II, or any combination thereof. In further embodiments, the insulin-like growth factor binding protein (IGFBP) includes but is not limited to IGFBP-3, IGFBP-5, or any combination thereof. In still further embodiments, the interleukin is selected from the group consisting of IL-8, IL-10, or any combination thereof.

In some embodiments of this invention, a hydrogel can be included in the scaffold, e.g., for long term delivery of biomolecules both in vitro and in vivo. Thus, in some embodiments of the invention, the scaffold further comprises a hydrogel.

In certain embodiments, a hydrogel of this invention can comprise extracellular matrix (ECM) molecules, such as thiolated ECM molecules. Such thiolated ECM molecules can include, but are not limited to, thiolated collagen, thiolated gelatin, thiolated laminin, thiolated fibronectin, thiolated heparin, thiolated hyaluronan (HA), any thiol group-containing peptide sequence, or any combination thereof. By using different ratios of these thiolated components and adjusting the cross-link density, a series of hydrogels can be formulated with a range of mechanical properties and customizable biomolecule release profiles. Thus, in some embodiments of the invention, the hydrogel can be a thiolated hyaluronan-collagen-fibronectin hydrogel. In other embodiments, the hydrogel can be a HA-gelatin hydrogel.

In some embodiments of the present invention, the hydrogel comprises one or more (e.g., 1, 2, 3, 4, 5, 6, 7, 8, 9, 10, 11, 12, 13, 14, 15, 16, 17, 18, 19, 20, etc.) biomolecule(s) of this invention in any combination. Thus, the biomolecules of the hydrogel include, but are not limited to, a differentiation stimulating biomolecule, a chemotaxis stimulating molecule, a proliferation stimulating biomolecule, a mobilization stimulating biomolecule, or any combination thereof, as described above.

It is also contemplated as part of this invention that the hydrogel can be contacted with the scaffold prior to and/or after the scaffold is delivered to the subject. Thus, the hydrogel can be associated with the scaffold prior to and/or post-implantation. The hydrogel can be introduced (“loaded”) into the scaffold by immersion or other contact of the scaffold with the hydrogel and/or the hydrogel's pre-gel constituents. The association of the hydrogel with the scaffold can be facilitated further by a physical means such as sonication or centrifugation. The hydrogel can be loaded by single or multiple contact events and/or injections and these contact events can occur pre- and/or post-implantation. The association between the scaffold and hydrogel can be temporary (e.g., no permanent fixation means used, may leak out over a period of time) or the association between the scaffold and hydrogel can be carried out by physically locking the hydrogel into place in the scaffold by hydrogel gelling and/or crosslinking post-loading (e.g., two completely independent but interpenetrating networks or IPNs without covalent linking between the two). The association between the scaffold and hydrogel can also be carried out by locking the hydrogel into place via induction (e.g., heat, etc), in which the hydrogel chemically interacts with the scaffold.

The present invention further provides methods of producing a scaffold of this invention. As one nonlimiting example, a method is provided herein of producing a scaffold comprising, consisting essentially of and/or consisting of one or more anti-inflammatory agents and comprising, consisting essentially or and/or consisting of one or more regenerative agents positioned on and/or within the scaffold to provide release first of the one or more anti-inflammatory agents into an environment of the scaffold, then release of the one or more regenerative agents into the environment of the scaffold, said method comprising: (a) producing a PLGA-collagen nanofibrous scaffold (e.g., by electrospinning techniques well known in the art); (b); crosslinking the scaffold of (a) (e.g., with genipin); (c) sterilizing the scaffold; d) depositing polyelectrolytes and one or more regenerative agents (e.g., BMP-2) into and/or on the scaffold to produce a deep layer (D), a double layer (DL) and a superficial layer (SF) on the scaffold; e) depositing a barrier nano-layer of genipin-crosslinked collagen to retard release of the regenerative agents from the scaffold; and f) depositing anti-inflammatory agents (e.g., colloidal SB203580 and/or PD98059 particles) onto the scaffold surface.

In certain embodiments of the invention, the scaffold produced according to the methods described herein can be treated, for example to wash off excess protein and such treating step can include boiling.

In further embodiments of this invention, one or more biomolecules are associated with the scaffold. Thus, the methods of this invention for producing a scaffold of this invention further comprise the step of associating one or more biomolecules with the scaffold. As stated above, the biomolecules, in the form of proteins, peptides and/or nucleic acids, can be incorporated into the scaffold at any step in the fabrication (pre-, during and/or post-fabrication) of the scaffold. Additionally, as noted above, in other embodiments, the biomolecules, in the form of proteins, peptides and/or nucleic acids, can be delivered directly to the subject according to well known methods.

The present invention additionally provides methods of first reducing or eliminating inflammation and subsequently regenerating or healing tissue (e.g., in a subject in need thereof), comprising contacting the subject with a scaffold of the present invention. In some embodiments the scaffold comprises, consists essentially of and/or consists of one or more biomolecules of this invention in any combination. In particular embodiments, the methods of regenerating tissue in the subject are carried out in the absence of cell transplantation that is recognized as part of the tissue regeneration process, either prior to, during or after contacting the subject with the scaffold. Specifically, tissue regeneration procedures known in the art include the transplantation of cells (autologous and/or allogeneic cells) into the subject and such cells facilitate the tissue regeneration process. The present invention is an unexpected improvement over such procedures, because the composition of the scaffold of this invention provides for the association therewith of one or more biomolecules that serve to attract the subject's own cells to the site where tissue regeneration is needed or desired, thereby obviating the need for transplanting cells (either autologous or allogeneic) into the subject as part of the tissue regeneration process.

As used herein, the terms “cell transplant” or “transplantation of cells” means the introduction from an external source of cells into a recipient. The cells can be the recipient's own cells that had been removed previously (i.e., autologous or homologous transplant) or the cells can be from a donor (i.e., an allogeneic, isologous or heterologous transplantation of cells not from the recipient).

Thus, the present invention provides a method of regenerating tissue in a subject, comprising contacting the subject with a scaffold of this invention, thereby attracting cells already present in the subject under natural conditions (i.e., not previously removed from the subject and returned to the subject as an autologous or homologous transplant) to the site of tissue regeneration and stimulating or activating said cells to regenerate tissue. In some embodiments, the subject may receive a cell transplant that is not a cell transplant that directly facilitates tissue regeneration.

Tissues that can be regenerated using this method include, but are not limited to, any hard or soft tissue, such as cartilage, bone, dental tissue, skeletal muscle, smooth muscle, skin, blood vessel, heart, liver, kidney, pancreas, brain, spinal cord, ligament, tendon, nerve tissue, etc., as would be well known in the art.

A site of contact for the scaffold of the present invention includes, but is not limited to, inside, outside, over, above, around, below, under and/or in proximity to a lesion, a wound, a diseased tissue, a joint space, a muscle, bone, connective tissue; an organ, a blood vessel, skin, a body cavity, etc., including any combination thereof.

Methods of contacting the subject in need thereof with the scaffold of the present invention include but are not limited to surgical implantation, placement into, on, around, inside, above, below, under, over and/or beneath a lesion, wound and/or body cavity, injection, topical delivery, or any combination thereof.

The term “subject” as used herein includes any subject in which inflammation reduction and tissue regeneration can be carried out. In some embodiments, the subject can be a mammalian subject (e.g., dog, cat, horse, cow, sheep, goat, monkey, rat, mouse, lagomorphs, ratites etc.), and in particular a human subject (including both male and female subjects, and including neonatal, infant, juvenile, adolescent, adult, and geriatric subjects, further including pregnant subjects). A subject in need thereof includes, but is not limited to, a subject having tissue that is and/or could become inflamed, injured, damaged, diseased and/or a subject that has or could develop an age related disorder associated with inflammation and tissue damage, degeneration, etc. and thus, is in need of and/or would benefit from and or desires inflammation reduction and tissue regeneration.

The term “therapeutically effective amount” or “effective amount,” as used herein, refers to that amount of a polypeptide, peptide, fragment, nucleic acid, virus, scaffold, nanoparticle, microparticle and/or composition of this invention that imparts a modulating effect, which, for example, can be a beneficial effect, to a subject afflicted with a condition (e.g., a disorder, disease, syndrome, illness, injury, traumatic and/or surgical wound), including improvement in the condition of the subject (e.g., in one or more symptoms), delay or reduction in the progression of the condition, prevention or delay of the onset of the condition, and/or change in clinical parameters, status or classification of a disease or illness, etc., as would be well known in the art.

For example, a therapeutically effective amount or effective amount can refer to the amount of a polypeptide, peptide, fragment, nucleic acid, virus, scaffold, microparticle, nanoparticle, composition, compound and/or agent (e.g., an anti-inflammatory agent, a regenerative agent, etc.) that improves a condition in a subject by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Treat,” “treating,” “treatment” or “healing” refers to any type of action that imparts a modulating effect, which, for example, can be a beneficial effect, to a subject afflicted with a condition (e.g., disorder, disease, syndrome, illness, traumatic or surgical wound, injury, etc.), including improvement in the condition of the subject (e.g., in one or more symptoms), delay or reduction in the progression of the condition, prevention or delay of the onset of the condition, and/or change in clinical parameters, disease or illness, etc., as would be well known in the art.

By the terms “treat,” “treating,” “healing” or “treatment of” (or grammatically equivalent terms), it is also meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder.

By “prevent,” “preventing” or “prevention” is meant to avoid or eliminate the development and/or manifestation of a pathological state and/or disease condition or status in a subject.

The present invention further provides delivering nanoparticles and/or microspheres comprising at least one biomolecule to the subject. Nanoparticles and microspheres comprising at least one biomolecule can be used for short-term biomolecule or signal delivery by encapsulating the biomolecule in nanospheres and/or microspheres. Material selection for the fabrication of the nanoparticles and microspheres and sphere diameter determines the length of the delivery period, as is well known in the art. Thus, in some embodiments, the nanoparticles and microspheres can be biodegradable. In other embodiments, the nanoparticles and/or microspheres can be nonbiodegradable. The nanoparticles and/or microspheres of this invention can be produced from any biocompatible material known in the art for such production.

The present invention further provides nanoparticles and/or microspheres comprising at least one biomolecule, wherein the at least one biomolecule is a biomolecule as described above. Accordingly, the biomolecule includes, but is not limited to, a differentiation stimulating biomolecule, a chemotaxis stimulating molecule, a proliferation stimulating biomolecule, a mobilization stimulating biomolecule, or any combination thereof, as described above. Other therapeutic agents or biomolecules that can be provided via the microspheres and nanoparticles include, but are not limited to, PNPX (para-nitrophenyl-beta-D-xyloside), cAMP, prolyl hydroxylase inhibitors (PHIs), and brain-derived neurotrophic factor.

The microspheres of the present invention can be in a size range of about 5 μm to about 50 μm. Thus, the microspheres can be 5 μm, 10 μm, 15 μm, 20 μm, 25 μm, 30 μm, 35 μm, 40 μm, 45 μm, 50 μm, and the like or any combination thereof. In other embodiments, the microspheres can be in a range from about 5 μm to about 10 μm, from about 5 μm to about 15 μm, from about 5 μm to about 20 μm, from about 5 μm to about 25 μm, from about 5 μm to about 30 μm, from about 5 μm to about 35 μm, from about 5 μm to about 40 μm, from about 5 μm to about 45 μm, from about 10 μm to about 15 μm, from about 10 μm to about 20 μm, from about 10 μm to about 25 μm, from about 10 μm to about 30 μm, from about 10 μm to about 35 μm, from about 10 μm to about 40 μm, from about 10 μm to about 45 μm, from about 10 μm to about 50 μm, from about 15 μm to about 20 μm, from about 15 μm to about 25 μm, from about 15 μm to about 30 μm, from about 15 μm to about 35 μm, from about 15 μm to about 40 μm, from about 15 μm to about 45 μm, from about 15 μm to about 50 μm, from about 20 μm to about 25 μm, from about 20 μm to about 30 μm, from about 20 μm to about 35 μm, from about 20 μm to about 40 μm, from about 20 μm to about 45 μm, from about 20 μm to about 50 μm, from about 25 μm to about 30 μm, from about 25 μm to about 35 μm, from about 25 μm to about 40 μm, from about 25 μm to about 45 μm, from about 25 μm to about 50 μm, from about 30 μm to about 35 μm, from about 30 μm to about 40 μm, from about 30 μm to about 45 μm, from about 30 μm to about 50 μm, from about 35 μm to about 40 μm, from about 35 μm to about 45 μm, from about 35 μm to about 50 μm, from about 40 μm to about 45 μm, from about 40 μm to about 50 μm, from about 45 μm to about 50 μm, and the like.

The nanoparticles of the present invention are in a size range of about 20 nm to about 50 nm. Thus, the nanoparticles can be 20 nm, 25 nm, 30 nm, 35 nm, 40 nm, 45 nm, 50 nm, and the like or any combination thereof. In other embodiments, the microspheres can be in a range from about 20 nm to about 25 nm, from about 20 nm to about 30 nm, from about 20 nm to about 35 nm, from about 20 nm to about 40 nm, from about 20 nm to about 45 nm, from about 20 nm to about 50 nm, from about 25 nm to about 30 nm, from about 25 nm to about 35 nm, from about 25 nm to about 40 nm, from about 25 nm to about 45 nm, from about 25 nm to about 50 nm, from about 30 nm to about 35 nm, from about 30 nm to about 40 nm, from about 30 nm to about 45 nm, from about 30 nm to about 50 nm, from about 35 nm to about 40 nm, from about 35 nm to about 45 nm, from about 35 nm to about 50 nm, from about 40 nm to about 45 nm, from about 40 nm to about 50 nm, from about 45 nm to about 50 nm, and the like.

The nanoparticles and/or microspheres of the present invention are delivered to the subject via a variety of methods, including, but not limited to, injection, surgical implantation, delivery into a body cavity, topical application, and any combination thereof. The nanoparticles and/or microspheres of this invention can be present in the scaffold of this invention and are therefore delivered to the subject via contacting of the subject with the scaffold. The nanoparticles and/or microspheres can also be delivered to the subject separately from the scaffold.

Embodiments of the present invention further provide a kit comprising one or more of the compositions described herein and optionally instructions for use and/or administration. It would be well understood by one of ordinary skill in the art that the kits of this invention can comprise one or more containers and/or receptacles to hold the reagents of the kit, along with appropriate reagents and directions for using the kit, as would be well known in the art. Each of these components of the kit can be combined in the same container and/or provided in separate containers.

The present invention is more particularly described in the Examples set forth below, which are not intended to be limiting of the embodiments of this invention.

### EXAMPLES

### Example 1

### Periodontal Disease

### Cytokines are Required for Periodontal Disease Progression.

In the oral microbial environment, bacterial constituents including Gram-negative derived lipopolysaccharide (LPS) can initiate inflammatory bone loss as seen in periodontal diseases. LPS can stimulate the expression of IL-1β, TNF-α, IL-6 and RANKL by activating the innate immune response¹⁻³. The production of inflammatory cytokines results from the activation of kinase-induced signaling cascades and transcriptional factors. LPS initiates this cascade by binding CD14 as well as toll-like receptors (TLRs), mainly TLR-2 and TLR-4⁴⁻⁶. Regardless of which TLR is engaged, LPS increases RANKL, IL-1, PGE₂ and TNF-α, each known to induce osteoclast activity, viability and differentiation⁷. In addition, activated monocytes, macrophages, and fibroblasts all produce cytokines such as TNF-α, IL-1β, PGE₂, and IL-6 within periodontal lesions^{8,9} and are significantly elevated in diseased periodontal sites compared to healthy or inactive sites¹⁰⁻¹⁴. These cytokines orchestrate the cascade of destructive events that occur in periodontal tissues and trigger the production of an array of inflammatory enzymes and mediators including matrix metalloproteinases (MMPs) and prostaglandins. Moreover, proinflammatory cytokines directly or indirectly recruit and activate osteoclasts through RANKL-dependent and independent pathways, resulting in irreversible bone destruction^{15,16}.

### Activation of Intracellular Signaling Pathways is Essential for Cytokine Production and Regulation.

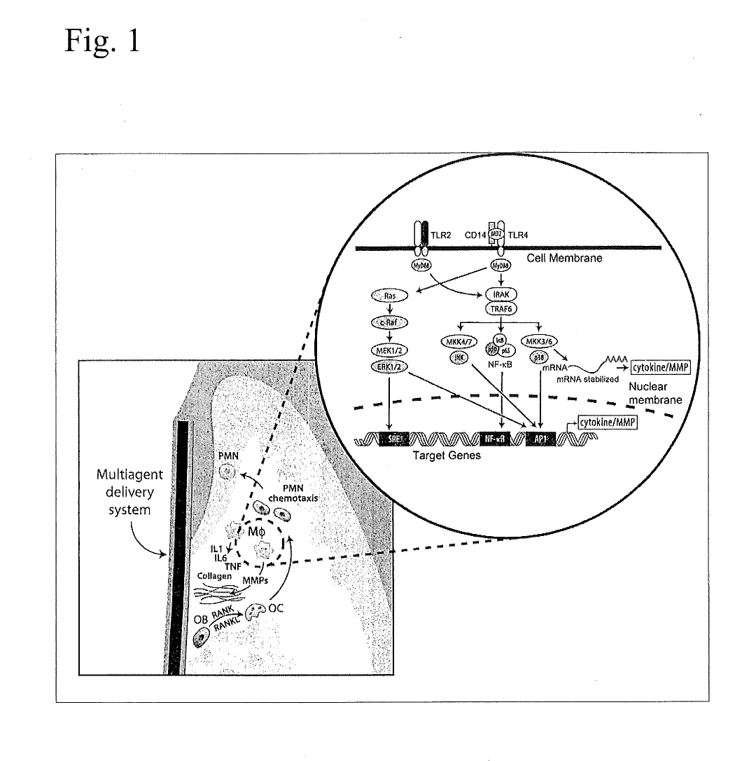

The innate immune system is the first line of defense against invading pathogens through a highly conserved pattern recognition system¹⁷. Innate immune cells, including macrophages and dendritic cells, express a series of Toll-like receptors (TLRs), which can bind to highly specific sequences expressed on microorganisms known as microbial-associated molecular patterns (MAMPS). Gram negative-derived lipopolysaccharide (LPS) initiates a potent inflammatory response cascade by binding CD14 (a cell surface protein) as well as Toll-like receptors (TLRs), mainly TLR-2 and TLR-4^{5,6}. Upon TLR-4 recognition of LPS, a complex series of orchestrated signaling events occur within innate immune cells, resulting in the production of cytokines that dictate the nature of the host response and mobilization of the adaptive immune response. Within periodontal tissues, TLR-2 and -4 expression is increased in severe disease states, suggesting that these receptors have an increased capacity to signal and influence downstream cytokine expression¹⁸. TLR-4 signaling activates MyD88-dependent pathways to subsequent activation of IRAK, TRAF6 and ultimately nuclear factor kappa B (NF-κB) that is required for cytokine induction. Also, TRAF6-dependent pathways are required for recruitment of different adaptor proteins and/or activation of various MAPK cascades such as ERK-1 and -2¹⁹, JNK and p38²⁰ needed for cytokine mRNA transcription and mRNA stabilization (FIG. 1; Table 1).

P. gingivalis and A. actinomycetemcomitans derived LPSs are considered key factors in the development of chronic periodontitis. LPS induction of disease leads to the initiation of a local host response in periodontal tissues that involves recruitment of inflammatory cells, generation of prostanoids and cytokines, elaboration of lytic enzymes and activation of osteoclasts²¹⁻²⁴. Activated monocytes, macrophages and fibroblasts all produce cytokines such as TNF-α, IL-1β, and IL-6 within periodontal lesions⁸. Bacterial LPS increases osteoblastic expression of RANKL, IL-1, PGE₂ and TNF-α; each known to induce osteoclast activity, viability, and differentiation^{7,25}. These cytokines orchestrate the cascade of destructive events through the production of an array of inflammatory enzymes and mediators including MMPs, prostaglandins and osteoclast recruitment and differentiation through RANKL-dependent and independent pathways, thus resulting in irreversible hard and soft tissue degradation^{15,16,26}.

MAPK Signaling Plays a Prominent Role in Regulation of Inflammatory Mediators.

The production of inflammatory cytokines is the result of receptor binding-induced signal transduction. Signal transduction pathways closely involved in inflammation include the activated protein MAPK pathway, phosphatidylinositol-3 protein kinase (PI3) pathway, janus kinase-signal transducer and activator of transcription (Jak-STAT), and NF-κB. Mitogen-activated protein kinases (MAPKs) are key enzymes in the signal transduction cascade of essentially every eukaryotic cell type²⁷. Major signaling pathways include ERK, JNK, and p38 MAPK. ERK is activated by mitogens and environmental stimuli while JNK, and p38 MAPK are activated by environmental stress and inflammatory cytokines^{28,29}. p38 MAPK plays a role in a variety of other cellular processes³⁰⁻³². Studies of both osteoblasts and chrondocytes have shown that the IL-1- or TNF-induced IL-6 production can be blocked with p38 MAPK inhibitors³³⁻³⁶. The functional consequence of blocking IL-6 was demonstrated further when p38 MAPK inhibitors were shown to prevent IL-1- or TNF-mediated bone resorption in an in vitro model³⁷. Orally-active p38 inhibitors have been shown to prevent and arrest periopathogenic LPS-induced bone destruction in a rat model^{38,39}.

p38 MAPK is a key intercellular signaling component of the innate immune system in macrophages. Activation of p38 following TLR engagement results in transcription factor activation (primarily AP-1 and NFκB). In addition, cytokine expression is enhanced following p38 activation through a transient increase in cytokine mRNA stability. Regulation of p38-induced mRNA cytokine stability is mediated via RNA-binding proteins (RNABPs, e.g., TTP) that bind to specific mRNA sequences called adenosine-uridine-rich elements (AREs)^{40,41}. These cis elements are located in the 3′ untranslated region (UTR) of many inflammatory cytokines (including TNFα, IL-6, and COX-2), conferring mRNA instability or translational silencing, thereby decreasing protein synthesis (FIG. 1).

However, in p38-stimulated cells, phosphorylation of TTP inhibits mRNA degradation and increases the production of proteins. Thus, RNABPs may serve as a target of cytokine-mediated inflammation. ERK1 and ERK2 are isoforms of the “classical” MAPK^{42,43}. Both ERK1 and ERK2 (referred to as ERK1/2) are activated by MAP/ERK kinase 1 (MEK1) and MEK2 (referred as MEK1/2), which are members of the MAPK kinase (MAPKK) family. MEK1/2 is activated by MAPK kinase kinase (MAPKKK)-mediated phosphorylation. These MAPKKKs include Raf and Mos. Activated MEK1/2 phosphorylates threonine and tyrosine residues in the Thr-Glu-Tyr (TEY) sequence of ERK1/2, resulting in the activation of ERK1/2. Activated ERK1/2 in turn phosphorylates many substrates including transcription factors, such as Elk1 and c-Myc, and protein kinase, such as ribosomal S6 kinase (RSK). Subsequently, immediate early genes, such as c-Fos, are induced. Since c-Fos and c-Jun constitute AP-1, an important transcription factor for the expression of many genes including inflammatory cytokines and MMPs, ERK1/2 are considered as crucial contributors to periodontal inflammation and tissue destruction.

LPS activates ERK1/2 in monocytes/macrophages and the ERK1/2 activation in turn upregulates inflammatory cytokines and MMPs^{44,45}. It has been shown that dominant-negative repressors of both Ras and c-Raf inhibited LPS induction of the TNFα promoter in RAW 264.7 macrophages⁴⁶, supporting a role of the Ras-c-Raf-MEK-ERK pathway in LPS-stimulated TNFα expression. It was also reported that LPS induction of MMP-1 production by monocytes is regulated by both ERK1/2 and p38, whereas MMP-9 production occurred mainly through the ERK1/2 pathway⁴⁵.

Analysis of the promoter regions of several MMPs, including MMP-1, -3, -7, -9 and -10, shows that these promoters contain the AP-1 binding motifs that locate at −68 to −80 relative to the transcription start site⁴⁷. A large number of studies have shown that PD98059 and U0126 inhibit MMP expression in macrophages^{48,49}, fibroblasts⁵⁰, endothelial cells⁵¹, and epithelial cells⁴⁸. Studies have also shown that simvastatin suppressed LPS-stimulated MMP-1 expression in macrophages by inhibiting ERK activity⁵². In animal studies, administration of PD98059 in a rabbit model of osteoarthritis decreased MMP-1 production by chondrocytes⁵³. All these studies indicate that the ERK signaling pathway-mediated AP-1 activation play a crucial role in inflammatory cytokine and MMP expression that may be critical in periodontal disease progression.

BMP-2 and PDGF are Crucial Regulators of Periodontal Tissue Maintenance and Regeneration.

The tissue microenvironment is influenced by several signals that aid tissues in maintaining a specific architecture. Growth factors have been recognized as a critical element in the maintenance, repair and regeneration of tissues, through their ability to induce proliferation, differentiation, matrix deposition and angiogenesis. Several growth factors, such as BMP-2, -4, -6, PDGF and IGF have been identified during embryologic development of periodontal tissue^{54,55}. Animal studies also demonstrated that these growth factors can improve periodontal tissue healing⁵⁶⁻⁵⁹. BMP-2 is a member of the TGF-β superfamily, and their production and receptors have been detected in both epithelial and mesenchymal cells during dental tissue development and adulthood^{55,60}. BMP-2 has been recognized for its ability to promote osteoblast differentiation and decrease proliferation of HPDL cells⁶¹.

BMP-2 has shown promise in clinically relevant dental and craniofacial applications. Superior alveolar ridge augmentation compared to controls was achieved when BMP-2 was used⁶². Platelet-derived growth factor (PDGF) plays an important role in embryonic development, cell proliferation, cell migration and angiogenesis, which is very important for periodontal tissue development, maintenance and regeneration⁵⁶. PDGF has been used to promote the regeneration of several periodontal tissues, including gingiva, alveolar bone and cementum⁶³. PDGF has a stimulatory effect on human periodontal ligament (HPDL) cells, which is important for periodontal ligament formation⁶⁴. These examples highlight the immense potential for regeneration of clinically functional periodontal tissues using PDGF and BMP-2. However, because of the short half-lives of the growth factors, bioengineering strategy is needed for developing local and sustained delivery of growth factors to achieve the desired therapeutic outcome.

Regeneration of periodontal tissues can only occur once the disease-associated inflammation has been addressed, which is attempted by removal/disruption of the dental biofilm that initiated the process. In the present invention, the inflammation associated with the disease process is arrested by using functionalized scaffolds to deliver small molecule inhibitors of cellular signaling to decrease the severity of the inflammatory response. Thus, this invention provides a completely novel bioengineering approach to periodontal regeneration, supported by the basic knowledge in inflammation control with the inhibition of ERK and the p38 pathway and by actively selecting the ideal cell population to the lesion area, followed by stimulation, differentiation and matrix production to regenerate periodontal tissue.

Conventional, clinically implemented approaches to periodontal regeneration focus on the use of barrier membranes to seal off rapidly proliferating gingival tissue in an effort to halt epithelial migration and encourage periodontal ligament reattachment⁶⁵. This concept of guided tissue regeneration helps to re-establish the organization of the junctional epithelium, however, it does not induce the recruitment or adhesion of osteogenic cells for regeneration of alveolar bone⁶⁶. Effective strategies for periodontal regeneration must incorporate several key parameters to produce properly organized neo-tissue. The bone marrow stem cells and periodontal fibroblasts provide two cell populations that are capable of providing cells that can regenerate lost periodontal tissues. Signaling molecules must be delivered in such a manner that cells are recruited and programmed to reach a specific destination. In addition, a vascular supply and mechanically apt scaffold must be present to provide a nutrient source and template for tissue formation^{67,68}.

Several approaches to periodontal regeneration and periodontal tissue engineering have been developed in attempts to overcome drawbacks of conventional strategies. Advancements in cell sourcing, scaffold fabrication, as well as growth factor and gene delivery have steadily increased. Regeneration of the periodontal ligament and other tooth supporting structures has been demonstrated through several scaffold-based strategies. Gelatin-chondroitin-hyaluron scaffolds seeded with autologous dental bud cells have been used to induce tooth formation in swine, with a 33% success rate of producing properly organized periodontal tissue⁶⁷. In another study, human periodontal ligament cells (HPLCs) seeded porous nanocrystalline hydroxyapatite/chitosan scaffolds led to both connective and vascular tissue ingrowth⁶⁹. Other types of composite membranes for guided tissue regeneration composed of both natural and synthetic polymers have been developed for potential periodontal regenerative treatment^{65,66,70,71}. Chitosan/collagen scaffolds incorporating TGF-beta1 gene and HPLCs have shown promise in producing regenerated periodontal ligament. The synergistic action of the scaffold and TGF-beta1 resulted in superior proliferation of HPLCs when compared to proliferation on the scaffold alone⁷².

Local delivery of growth factors from matrices that precisely control release offer promising approaches to heal periodontal defects. For example, platelet derived growth factor (PDGF) release from beta-tricalcium phosphate carriers promoted the local production of vascular endothelial growth factor (VEGF) in wound fluid of patients with localized periodontal defects⁷³. Release of insulin-like growth factor-I (IGF-I) from dextran-gelatin microspheres resulted in formation of new bone, cementum and periodontal ligament in vivo⁷⁴. There are drawbacks with currently tested bioengineering strategies, since these strategies are only addressing one issue of the periodontal regeneration, i.e., growth factor delivery and cell components. There is a critical need to control inflammation before regeneration can occur properly, which has to be tested in a true periodontal disease model.

While use of a periodontal defect model is an important first step in determining treatment efficacy, these approaches failed to incorporate a true periodontal disease model. It is essential that the developed approaches are tested under conditions in which chronic microbial contamination and subsequent tissue destruction persist. To follow, these strategies fail to incorporate a method through which microbial contamination can be harnessed, so that complete and predictable periodontal regeneration is attained. Thus, there is a need to develop novel bioengineered approaches to periodontal regeneration that provide local delivery of bioactive factors, incorporate structurally apt yet biodegradable scaffolds, induce angiogenesis, and suppress the immune-host response to microbial contamination such that new periodontal tissue is reliably produced.

### Recruitment of Postnatal Stem Cells for Periodontal Regeneration.

The traditional concept of cell therapy is based upon several basic steps. The first step is cell sourcing and cells may be isolated from autologous, allogenic, or xenogenic sources. Second, the isolated cells are expanded in vitro to a cell population sufficient for effective treatment. The expanded cells can also be seeded on a scaffold and cultured in a bioreactor. Finally, the expanded cells are re-implanted into the patient. However, this final process is associated with ethical, economic, regulatory and clinical problems. Clinically, allogenic and xenogenic sources face the greatest likelihood if immune rejection by the patient. Ethical and regulatory issues must also be resolved for this to be a routine clinical treatment. Thus, autogenous cells would seem to be the best choice, but cell isolation from patients in need of treatment can cause additional normal tissue morbidity. In order to obtain a sufficient number of cells for transplantation, in vitro proliferation is essential, which may cause undesirable phenotype changes⁷⁵. Pluripotency of stem cells may decrease during in vitro culture^{76,77}. Allogenic and xenogenic components used in culture may cause host immune rejection. In addition, the cost for in vitro expansion of stem cells is very high, since a battery of growth factors is needed for the propagation procedures. The economic aspects and multi-week expansion period present important challenges to these clinical procedures. Finally, while mesenchymal stem cells attract much attention due to their pluripotency, the pluripotency of mesenchymal stem cells decreases during in vitro culture using conventional 2-D culture conditions^{76,77}.

An alternate cell source could be endogenous stem cells. There are several advantages to the use of endogenous stem cells for tissue repair. First, using endogenous stem cells avoids the immunocompatibility issues that accompany the use of allogenic and xenogenic cells. Second, it is easier, safer and more efficient to use endogenous stem cells for tissue repair to expand and re-implant autologous cells. Third, only a single surgical intervention is required, rather than two surgeries several weeks apart. Finally, the process of recruiting endogenous stem cells offers both regulatory and economic advantages relative to ex vivo approaches. The present invention provides for enhancement of the recruitment of endogenous stem cells into the lesion site for periodontal and skin tissue regeneration. Several factors, such as hepatocyte growth factor (HGF)⁷⁸, stromal cell-derived factor-1 (SDF-1)⁷⁹, PDGF, BMP-2, and BMP-4⁸⁰, have been shown to attract bone-marrow stem cells. However, like most proteins, BMP-2, PDGF and other growth factors undergo rapid proteolysis in vivo, resulting in a very short lifetime of the bioactive growth factor. The half-life of BMP-2 and PDGF delivered in a soluble form in vivo is less than 20 minutes⁸¹. In contrast, the time required to recruit a sufficient number of stem cells and induce them to the appropriate phenotypes for tissue repair is usually days to weeks. Therefore, direct injection of growth factors to the repair site has limited success. To maintain the therapeutic level of growth factors at the repair site necessary for endogenous stem cell recruitment, sustained, long-term and localized delivery of PDGF, BMP-2 and other such bioactive agents is essential. Several polymer delivery systems are being developed for proteins and growth factors delivery. Reservoir devices, solid implants, polymeric micro- and nano-particles, and hydrogels are the most commonly used. Polymer systems have many advantages; for example, they can stabilize proteins, provide localized delivery, and produce diffusion-limited concentration gradients in tissues. ECM-based scaffolds with a wide array of physiological functions represent ideal substrates for PDGF and BMP-2 delivery and stem cell recruitment and differentiation, since ECM based materials may provide adhesion sites for migrating stem cells to grow in. In one aspect of the present invention, a degradable scaffold (e.g., a polymer-collagen hybrid scaffold) combined with the layer-by-layer system described herein will be used for long-term delivery of bioactive agents such as growth factors. Thus, in a particular, exemplary embodiment of this invention, scaffold-delivered antimicrobials and/or p38/ERK inhibitors will be used to control infection and/or inflammation while sequentially and spatially delivered PDGF/BMP-2 will be used to regenerate LPS-induced experimental periodontal tissue loss. For example, for diabetic ulcer, one or more antimicrobial layers, one or more anti-inflammation layers and one or more regeneration layers [comprising, consisting essentially of or consisting of e.g., platelet derived growth factor (PDGF), epidermal growth factor (EGF), fibroblast growth factor (bFGF), granulocyte macrophage colony stimulating factor (GM-CSF), keratinocyte growth factor-2 (KGF-2) and/or transforming growth factor beta (TGF-β) in any combination].

A. actinomycetemcomitans LPS can Potently Induce MAPK Signaling.

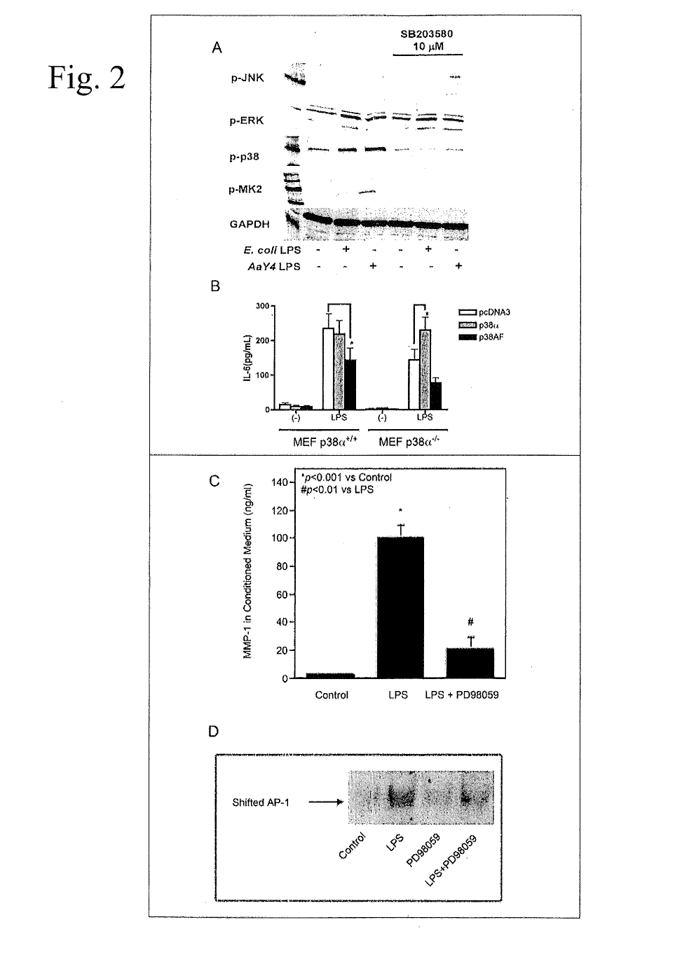

A. actinomycetemcomitans LPS has been used to characterize MAPK signaling intermediate activation in a variety of periodontal tissues including osteoblasts, periodontal ligament fibroblasts and macrophages^{39,82,86,88}. As shown in FIG. 1A, A. actinomycetemcomitans LPS induced primarily p38 MAPK in 15 min stimulation experiments. In addition, JNK kinase was also activated. Mitogen activated protein kinase-activated protein kinase-2 (MK2), a downstream kinase from p38 MAPK that actively participates in mRNA stability regulation, is also activated by A. actinomycetemcomitans LPS. In addition, ERK signaling is shown to be required for MMP-1 expression (FIG. 2, lower panel) and AP1 activation in monocytes. Also, MMP-13, IL-6, and RANKL have been shown to require p38 MAPK signaling in periodontal ligament fibroblasts^{39,82,85,86,88,98}. Collectively, these data support the concept that multiple MAPK signaling is necessary to control LPS-induced cytokine and MMP expression in the periodontal microenvironment.

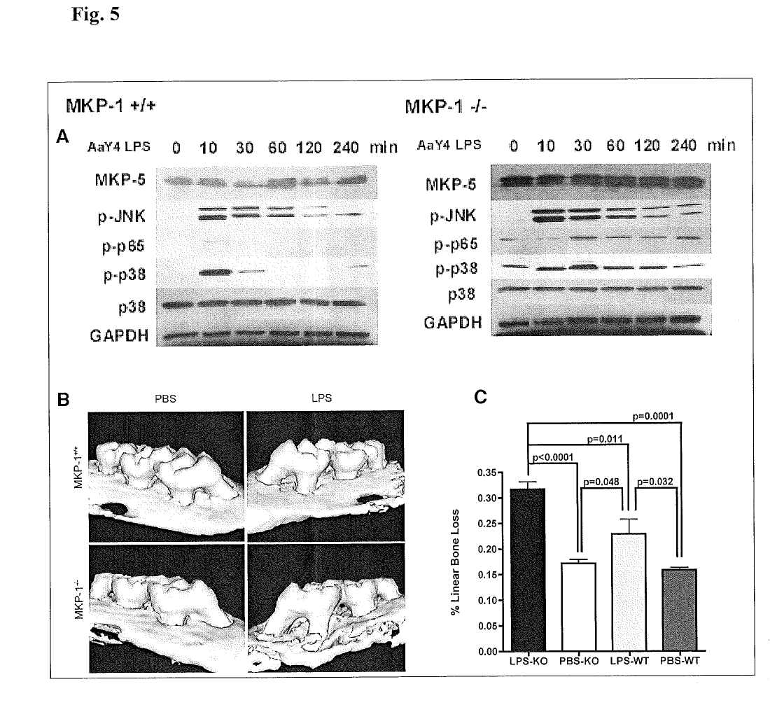

Furthermore, LPS-induced IL-6 mRNA expression has been shown to require multiple MAPK signaling pathways including p38⁸⁸. However, p38 signaling mediates mRNA stability through activation or inactivation of ARE-BPs. Notably, p38/MK2 signaling has been shown to phosphorylate tristetraprolin (TTP) to inactivate this destabilizing ARE-BP by sequestering the ARE-BP complex away from stress granules where RNA decay is likely to be initiated. The significance of TTP regulation of ARE-containing transcripts (e.g., IL-6) was demonstrated in vivo in studies wherein overexpression of TTP in an experimental model of periodontal disease resulted in the arrest of A. actinomycetemcomitans LPS-bone loss⁹⁹. In related studies of signaling mechanisms necessary for periodontal disease progression, the negative regulator of p38 and ERK signaling, MAPK phosphatase (MKP)-1 was shown to be required to attenuate MAPK signals in inflammatory bone loss models (described below). This occurs both in vitro (FIG. 5) where sustained activation of p38 signaling occurs in the absence of MKP-1 in bone marrow stromal cells. JNK signaling is not affected by the lack of MKP-1 in this model. These studies have been continued in the whole animal to demonstrate the importance of these signals in the LPS-induced periodontal bone loss model, where MKP-1 mice lose significantly more bone as compared to wild type control mice (FIGS. 5B-C).

p38 MAPK Signaling is Required to Mediate A.a. LPS Periodontal Bone Loss.

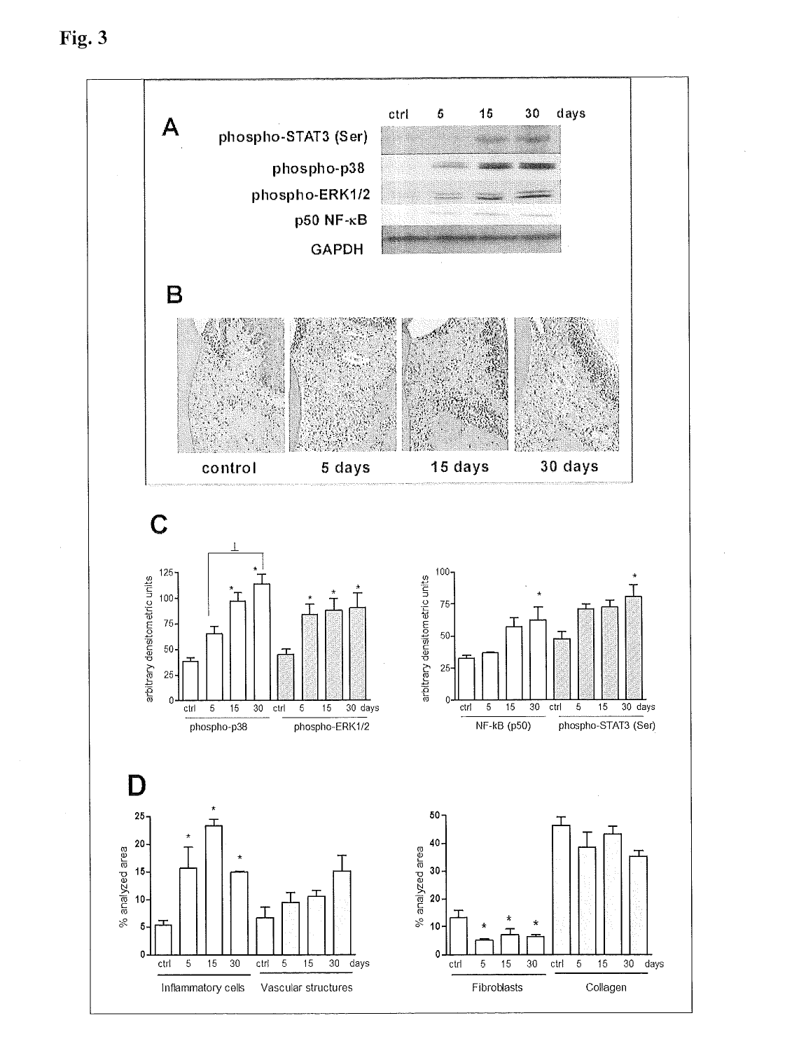

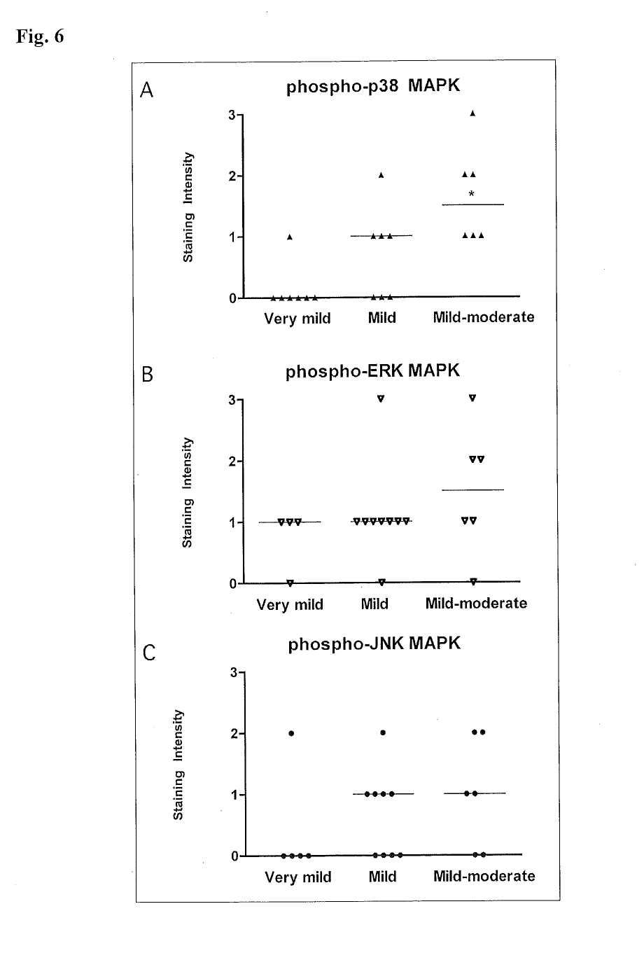

Several different experimental periodontitis animal models have been established, including a straightforward model of LPS-induced bone loss. This type of model exhibits many features of human disease including bone resorption due to osteoclastic activity and cytokine production¹⁰⁰⁻¹⁰³. Consistent with human pathology, data obtained from A. actinomycetemcomitans LPS-induced bone loss has indicated excessive proinflammatory cytokine production and osteoclast-induced bone loss (FIG. 3; ¹⁰⁴). This model has proved to generate consistent bone loss following microinjection of LPS over a 4-week period (mean linear bone loss of 0.9967 mm [SD=0.24 mm, coef. of variation=24.11%; n=6]). Activation of phospho-p38MAPK has been demonstrated in this model³⁸. These data have been expanded to human periodontal disease pathology where both phospho-p38 and to a lesser extent phospho-ERK but not phospho-JNK appear to be correlated with clinical disease severity and inflammation (FIG. 6). Interestingly, phospho-JNK levels did not correlate with the degree of inflammation. This model is employed in studies described herein to demonstrate control of LPS-induced periodontal inflammation while using engineered scaffolds to release small molecule inhibitors and growth factors in a temporally controlled manner to regenerate lost periodontal structures.

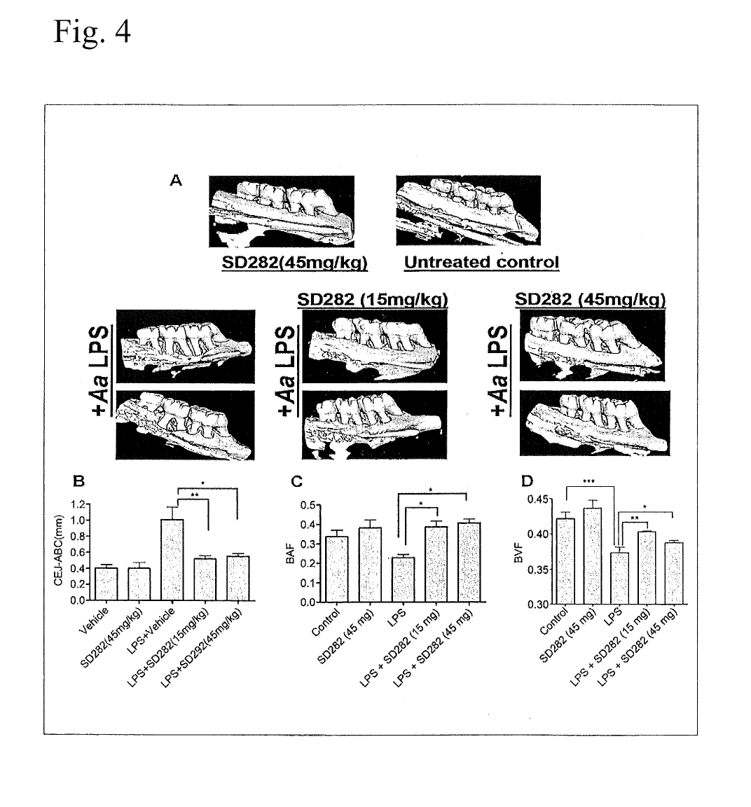

Two major lines of evidence indicate the significance of p38 MAPK signaling in periodontal disease progression. The first evidence comes from the ability of an orally active p38α inhibitor (SD-282; Scios, Inc.) to prevent A. actinomycetemcomitans LPS-induced experimental periodontitis in a rat model (FIG. 4 and³⁸). In addition, p38 inhibitors arrested LPS-induced bone loss from after bone loss was established³⁹. The second line of evidence comes from the MKP-1 null mouse, in which A. actinomycetemcomitans LPS-induced alveolar bone loss is more profound that in wild-type control mice (FIG. 5B). Because MKP-1 dephosphorylates p38 and ERK/JNK in some cases, these data provide strong evidence that p38/ERK signaling is a vital component in LPS-induced events in the periodontal environment. In both cases, the extent of alveolar bone loss and periodontal disease destruction was assessed by μCT.

Data obtained from human periodontal disease samples indicate that activated (phosphorylated) levels of p38 and ERK are elevated as compared with healthy controls. Samples were taken from areas of periodontal surgery after initial periodontal therapy (post scaling and root planing), whereas healthy samples were from implant or pre-orthodontic crown exposure surgeries. All clinical parameters were evaluated including plaque index, pocket depth, clinical attachment loss, periodontal index, and BANA (for microbiological index). As shown in FIG. 6, phospho-p38 levels correlated with periodontal disease severity (as measured by periodontal index) and approached significance with phospho-ERK. All other periodontal parameters correlated with phospho-p38 (except the plaque index). These data support data obtained from small animal models of periodontal disease and highlight the significance of regulating these MAPK pathways for control of periodontal inflammation.

### Fabrication of Biodegradable Nanofibers Using Electrospinning Technology.

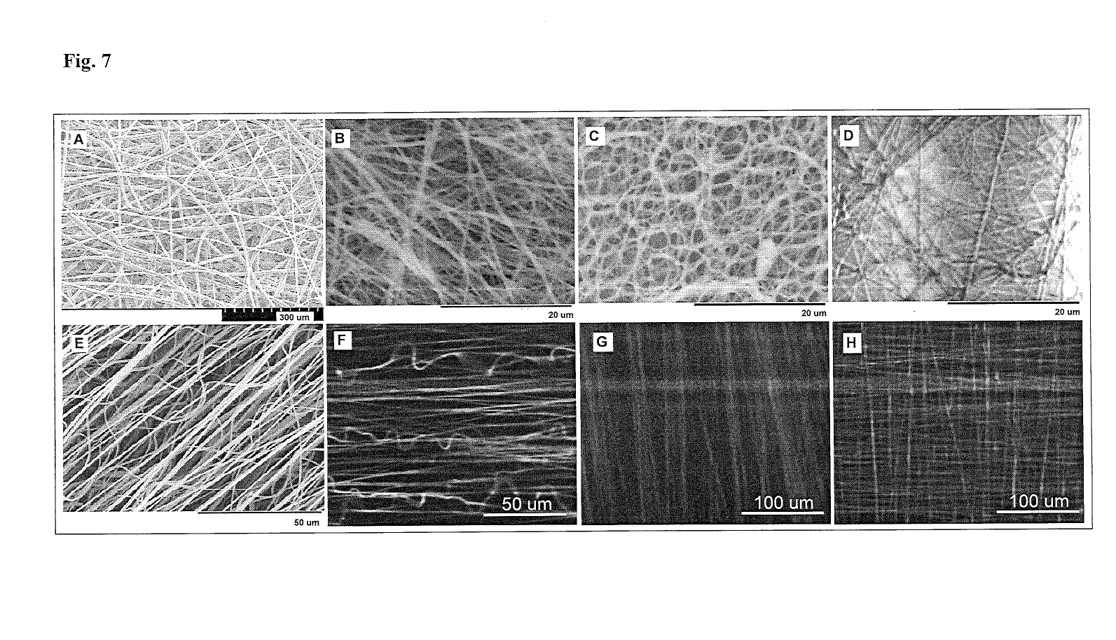

Degradable synthetic polymers, such as PLGA, polycaprolactone (PCL), polyurethane (PU), and natural polymers such as collagen, gelatin and/or chitosan can be dissolved in the appropriate solvents and at a concentration ranging from about 1% to about 20% wt/v. Polymer solutions are fed by syringe pump at a controlled flow rate through a blunt tipped needle. A voltage of about 1 KV to about 30 KV is applied to the needle tip with a high voltage power supply. The needle tip is held at a certain height above collecting devices designed to collect nanofibrous scaffolds. By varying the collecting techniques and feeding conditions, degradable nanofibers of different materials (FIGS. 7A-D) and different patterns (FIGS. 7E-H) have been fabricated.

### Loading of p38 or ERK Inhibitors and BMP-2 on PLGA-Collagen Scaffold Using Nano-LbL Technique.

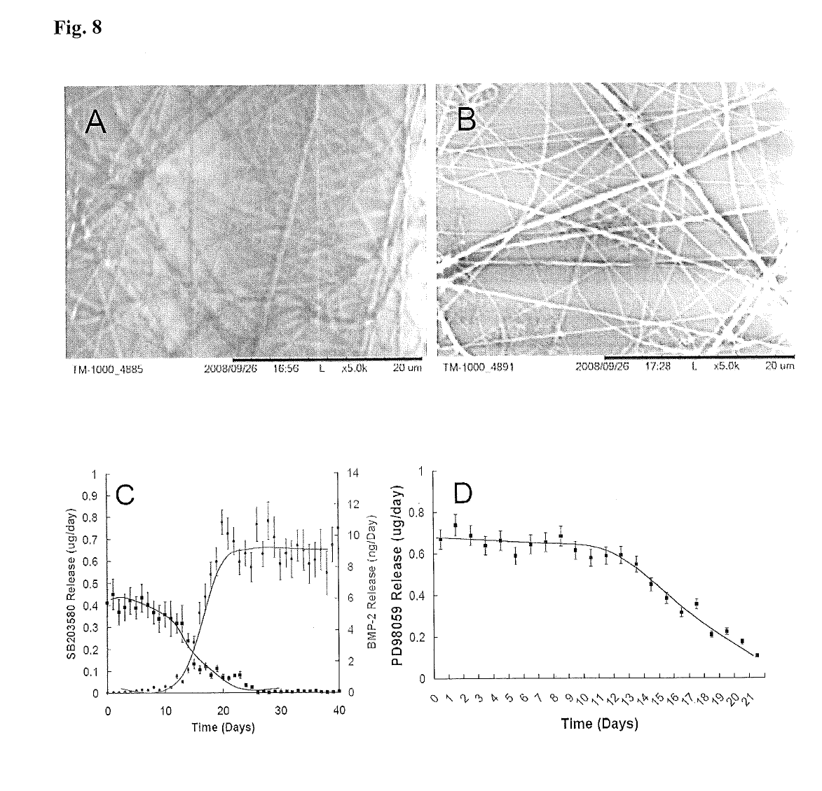

Nano-Layer-by-Layer (NanoLbL) technology is used to further functionalize biodegradable scaffolds. Using this technique, surface modification of a charged template occurs via electrostatic interactions. It offers a facile method to create multifunctional nano-coatings with tunable release kinetics. A compartmentalization technique that allows for multimolecule release was used. In one example, PLGA 50:50 copolymer and collagen were dissolved in hexafluoro-2-propanol in a ratio of 7 to 1. The polymer solution was fed by syringe pump at a rate of 0.015 mL/min through a 23 gauge blunt tipped needle. A voltage of 10 kV and a working distance of 10 cm were used. The PLGA/collagen fibers were collected on square glass sheets on top of grounded aluminum foil. The fibrous scaffolds are then crosslinked in 5% genipin solution for one hour. The scaffolds were rinsed in ethanol and dried in a stream of nitrogen prior to deposition of nano-layers. Collagen, having an isoelectric point of 5.5, is negatively charged at physiologic pH, providing a charged surface upon which the nanoLbL process can proceed. Polyelectrolyte solutions of polycation poly (allylamine hydrochloride) (PAH), and the polyanion poly(acrylic acid) (PAA) were prepared in 0.15M NaCl solution at 1 mg/mL. BMP-2 was reconstituted in 4 mM HCl with 0.1% human serum albumin content at 10 ug/mL. Deposition times for PAH and PAA and growth factors such as BMP2 were ten minutes, followed by rinsing in ultrapure water. Three loading architectures were used to incorporate growth factor, such as BMP-2 in the bottom compartment of the coating. These architectures, deep (D), double-layer (DL) and superficial (SF), correspond with the position of BMP-2 within the lower compartment of the nanoLbL coating. Thus, the following layering schemes were used: Deep layer with [BMP2-(PAH/PAA)₃], double layer with [(PAH/PAA)-BMP2}₂-(PAH/PAA)], and superficial layer with [(PAH/PAA)₃-BMP-2]. Then a barrier nano-layer of genipin-crosslinked collagen was used to retard release of BMP-2 from the lower compartment of the film. Following deposition of the barrier layer, SB203580 (a p38 inhibitor) and/or PD98059 (an ERK inhibitor) were deposited onto the film surface for ten minutes. Both SB203580 and PD98059 are hydrophobic, and have estimated isoelectric points between 5 and 6, rendering them negative at physiologic pH. NanoLbL assembly is an aqueous process, so to incorporate SB203580 and/or PD98059 into the film assembly, SB203580 and/or PD98059 were added to PAH at 0.1 to 2 mg/mL and sonicated for 30 minutes to create positively charged, soluble particulate aggregates. The aggregates were centrifuged at 10,000 rpm for five minutes, rinsed with ultrapure water, and resuspended in either 0.15M NaCl or water. A deposition time of ten minutes, followed by rinsing in ultrapure water was used for adsorption of the SB203580 and/or PD98059 aggregates. The scaffolds were placed in 12-well dishes for release testing. A volume of 1 mL PBS was used as the release medium. After each 24 hours, 100 uL samples were removed and replaced with fresh PBS. Samples were kept at −20° C. until needed for analysis. The release profiles of PD98059 and SB203580 were determined using UV-vis spectroscopy and the release profile of BMP-2 was determined using ELISA. Samples from each experimental group were thawed and plated in 96-well format. The amount of PD98059, SB203580 and BMP-2 was quantified based on a standard concentration curve at the peak wavelengths of 260 nm and 315 nm, respectively. For SB203580 and BMP-2 loaded samples, the release was monitored for up to 40 days. For PD98059 loaded samples, release was monitored for 21 days. As shown in FIG. 8, after nanoLbL coating on the surface of PLGA-collagen nanofibers, the scanning electron microscope (SEM) image is different, due to the charged surface of the coating layers. All SEM samples are not sputter coated, since the Hitachi TM-1000 SEM can detect uncoated samples. The release profile of inhibitors is stable for about two weeks (FIGS. 8C-D) and the release of BMP-2 from the bottom compartment started after release of the inhibitors (FIG. 8C). Monitoring of release has been carried out for up to 40 days; however the BMP-2 release may last much longer. Importantly, using this nanoLbL approach, the length of the release can be well controlled by controlling the thickness of the coating or the length of the coating process.

p38 and ERK Inhibitors and BMP-2 Released from NanoLbL Coating on PLGA-Collagen Scaffold Retain Pharmacological Activity In Vitro.

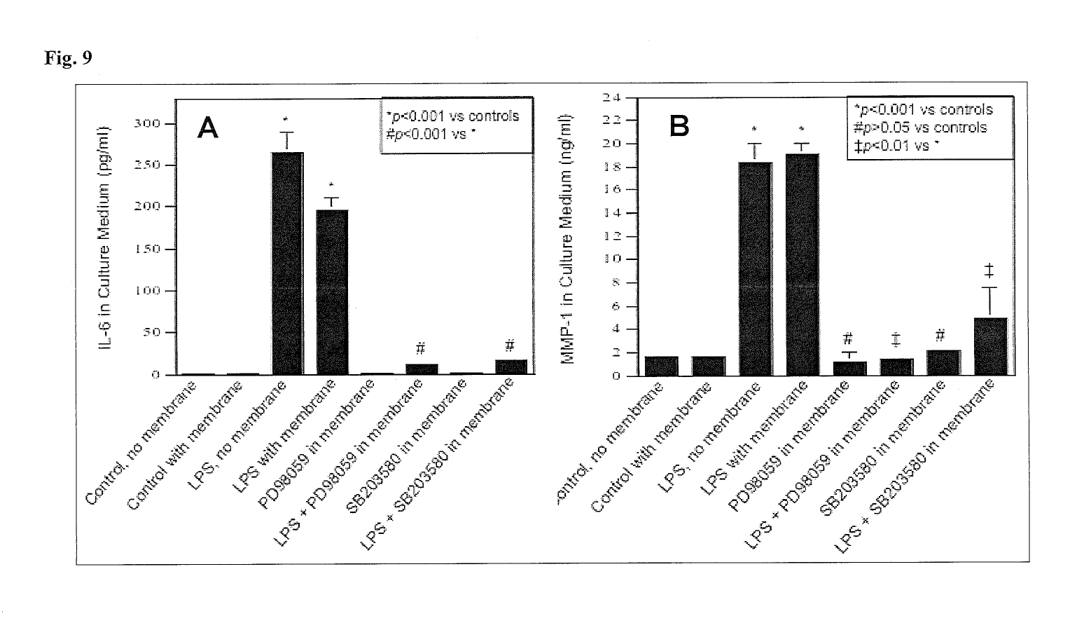

Data from the studies described herein indicate that the scaffolds of this invention can release the p38 inhibitor (SB203580) (FIG. 8C) or the ERK inhibitor (PD98059) (FIG. 8D) into tissue culture medium and retain biochemical activity. As shown in FIG. 9, LPS stimulated either IL-6 (A) or MMP-1 (B) in cultured macrophages with or without the PLGA-collagen scaffold present. Both PD98059 and SB203580 retain their pharmacological activity, showing significant inhibition of both IL-6 and MMP-1 in LPS stimulated cultures.

Ectopic Bone Formation with Scaffold-Loaded with BMP-2.

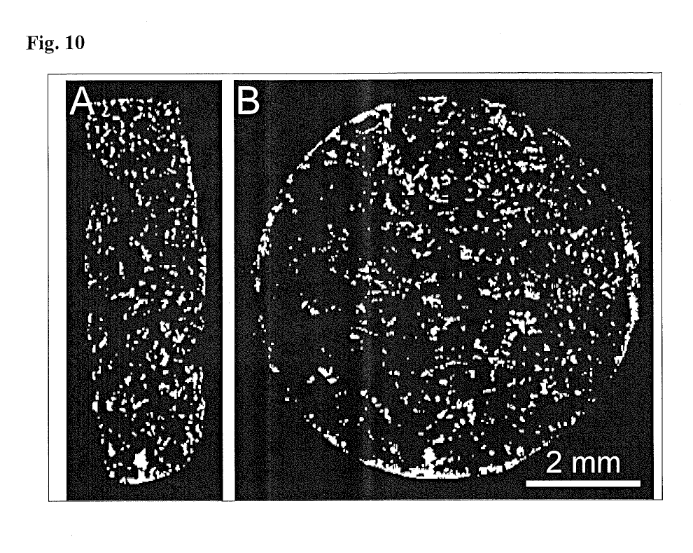

Using the same nanoLbL technology, BMP-2 was loaded onto scaffolds of 5 mm diameter and 2 mm thickness. The scaffolds were implanted subcutaneously into the backs of adult male S.D. rats. After 4 weeks, the rats were sacrificed and the implants were harvested for μCT examination for bone formation inside the scaffolds. These in vivo data show that BMP-2 can induce ectopic bone formation from a nanoLbL scaffold (FIG. 10).

### Summary of Supporting Data

The studies described above demonstrate that 1) p38 MAPK and or ERK signaling is essential for IL-6, MMP-1, MMP-13, and RANKL expression and production; 2) A. actinomycetemcomitans LPS induces alveolar bone loss in rat models of experimental periodontitis; 3) p38 MAPK signaling is required for A. actinomycetemcomitans LPS mediated bone loss; 4) nanolayered PLGA-collagen nanofibrous scaffolds can release p38 and ERK inhibitors to decrease LPS-stimulated IL-6 and MMP-1 production in monocytes; and 5) scaffold-loaded BMP-2 is capable of forming bone in vivo.

Optimizing the Delivery Kinetics of p38 MAPK or ERK Inhibitors from the Nanolayer Coating on the Surface of PLGA-Collagen-Based Scaffolds to Control LPS-Induced Inflammatory Cytokines In Vitro and In Vivo.

Previous data have indicated that p38 signaling is required for LPS-induced bone loss^{38,39}. Additional preliminary data from MKP-1 null mice is in agreement with inhibitor data (see FIG. 5). LPS also induces MMPs in the periodontal microenvironment that require both p38 and ERK signaling for maximal expression. Importantly, cross-talk between p38 and ERK MAPK pathways implies that inhibiting p38 MAPK can result in activation of ERK^{87,105,106}. However, this type of cross-talk has not been shown with JNK. This supports the strategy of simultaneous inhibition of both p38 and ERK; and also supports the strategy of transient inhibition of these signaling pathways, since the sustained inhibition of these pathways can result in feedback-activation of NF-κB with the associated increase in the expression of genes involved in inflammation and immune response¹⁰⁶. Collectively, these data suggest that the combination of p38 and ERK inhibitors may be more efficacious for inhibition of LPS-induced cytokine and MMP production.

### In Vitro Validation of Scaffold-Released Inhibitors.

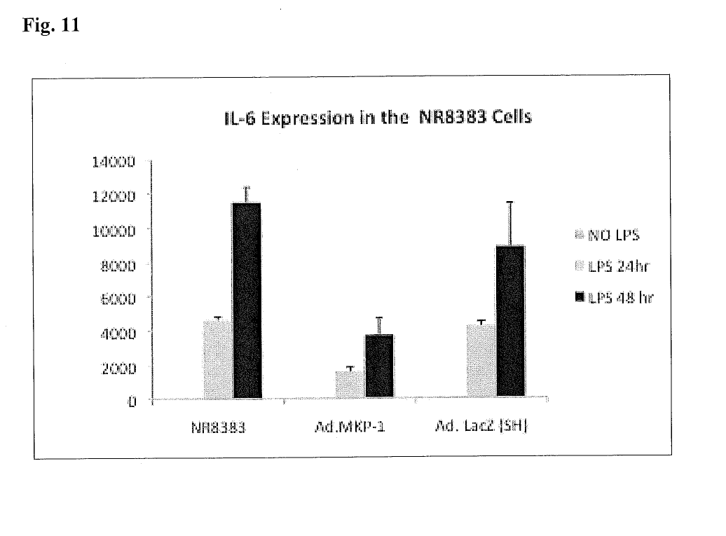

A rat monocyte/macrophage cell line (NR8383; ATCC CRL-2192) will be used for these in vitro studies. Cells from this cell line are both adherent and in suspension, permitting an evaluation of cytokine expression in cultures containing scaffolds impregnated with inhibitors. Cells will be plated in 6-well culture dishes containing PLGA-collagen-based scaffolds with nanoLbL coating loaded either SB203580 (p38 inhibitor; 11-50 μg each scaffold in 5×3 mm dimension, which is the scaffold size used for implantation), PD98059 (ERK inhibitor; 11-50 μg each scaffold in 5×3 mm dimension) alone or in combination. These cells respond to A. actinomycetemcomitans LPS to generate IL-6 in a p38/ERK dependent manner (FIG. 11). These cultures will be stimulated with A. actinomycetemcomitans LPS with cell culture supernatants and cytoplasmic RNA will be collected 24, 48, and 72 hours after A. actinomycetemcomitans LPS stimulation to assess IL-6 and TNF-α levels and IL-6 and TNF-α mRNA expression, respectively. In a separate series of experiments, whole cell lysates from NR8383 cells will be analyzed by immunoblot analysis to determine the specific effects of p38/ERK inhibitors on short-term stimulation (0-240 min) by LPS. Phosphorylated forms of p38, JNK, and ERK MAP kinases will be evaluated compared to non-phosphorylated controls (similar to studies described in FIG. 2). From these initial series of experiments, it is expected that macrophages will have a variable degree of attenuated level of cytokine expression in stimulated cultures compared to ‘empty’ scaffold controls. Both p38 and ERK inhibitors (0.5-5 μg/ml each) will be used as controls for cytokine (TNF and IL-6) inhibition. The intent from these studies is not to establish the release kinetics for in vivo usage, but rather to demonstrate that in short-term stimulated monocyte/macrophage cultures, scaffold-released p38/ERK inhibitors have pharmacological activity. In the event that no LPS-induced cytokine expression is observed, such data may indicate that the inhibitors are not released at high enough concentrations from the scaffolds to inhibit cytokine expression. Alternatively, this may indicate that inhibitors may be released with much slower kinetics than LPS-induced cytokine translation (as measured by ELISA).

Once initial parameters are established for p38 and ERK inhibitors independently in PLGA-collagen-based scaffolds, optimal combinations of these inhibitors will be identified by evaluating the ability of various combinations of inhibitors to attenuate IL-6 and TNF. Since both signaling pathways are involved in the generation of these cytokines in response to LPS, at least an additive effect in vitro is expected with respect to inhibition of cytokine expression. These studies will be expanded to isolate cytoplasmic mRNA to evaluate changes in cytokine and MMP expression from control and p38/ERK inhibitor scaffold cultures. Cytokines to be evaluated are TNF, IL-1, IL-6, IL-8, IL-10, and COX-2. MMP-1, -3, and -13 will be evaluated by real-time qRT-PCR as described herein. Data will be quantitated and expressed as fold change compared with 18S ribosomal RNA. Many of the above named transcripts have short-lived mRNAs and therefore it is important to determine if the inhibitors are regulating the signaling pathways needed for both transcriptional and post-transcriptional regulation to rule out effects specific to the half-life of the protein.

### In Vivo Evaluation of Inhibitor-Containing Scaffolds.

Using the established model of inflammatory bone loss, A. actinomycetemcomitans LPS can predictably generate alveolar bone loss upon repeated injection. For these studies, LPS will be used to induce inflammation and bone loss for 2 weeks. After inflammation induction, mucoperiosteal flaps will be raised on the palatal aspect of the molar region of anesthetized rats. Mock controls (nano-structured biomaterials (NSB) with and without inhibitors) will be surgically placed. Upon scaffold placement, surgical sites will be closed with n-butyl-cyanoacrylate (Vetbond). LPS will continue to be microinjected until sacrifice. Animals in each group will be sacrificed at 1, 3, 7, and 14 days after implantation of scaffold (n=6/time period/group). Since LPS injections will be performed bilaterally, half of the samples harvested at each period will be used for IHC and histological analysis and half will have the soft tissues harvested for the extraction of total proteins for quantitation by multiplex bead-based assays (Bio-Plex 200 Suspension Array System, Bio-Rad Lab.). This multiplex analysis allows an assessment of the expression of a panel of inflammatory cytokines (IL-1α, IL-1β, IL-2, IL-4, IL-6, IL-10, GM-CSF, IFN-γ and TNF-α-Bio-Plex Rat Cytokine 9-Plex A Panel, Bio-Rad Lab) that will provide a good overview of how the inflammatory process was affected by the scaffolds. Also, in these same samples, the same methodology will be used to study the activation of multiple signaling pathways by quantitation of phosphorylated forms of p38, JNK, ERK1/2 and IκB-α normalized to the quantities of the total forms of these same proteins using custom-mixed multiplex assays for phosphoprotein detection (X-Plex assay service, Bio-Rad Lab). This approach maximizes the use of samples, reducing the number of animals needed in the experiment and increases the throughput, allowing a more comprehensive analysis of cytokine expression and signaling profile.

It is estimated that 6 rat maxillas per treatment group will be needed. This is based upon power calculation assuming a sample size of 6, standard deviation of the outcome measure of 0.42 units and 6 levels of treatment (groups), then a difference (contrast) of 1 unit of measurement would be detected at an alpha error of 0.05 with a power of 0.81 by One-way ANOVA with post-hoc Bonferroni pairwise comparisons. For these studies, there will be 6 groups and 2 treatment groups containing LPS with scaffolds (+/−inhibitors). The control group of no LPS and no scaffolds will also have a mock surgery to control for surgical wound induction of inflammation/cytokine responses. If these studies indicate that scaffold-released inhibitors would be able to inhibit LPS-induced bone loss in this model, such data would be interpreted to indicate that pharmacological inhibitors can be successfully delivered to the periodontal microenvironment to control inflammation and concomitant bone loss. Failure to observe significant differences with scaffolds containing inhibitors (in LPS treated groups) may indicate that release kinetics obtained from in vitro study is not optimal for this in vivo model.

Establishing the Appropriate Delivery Profile of BMP-2 and PDGF in a Spatial Distribution Pattern from a Nanolayer Coating on PLGA-Collagen Based Scaffolds

The scientific rationale which represents the current paradigm of periodontal regeneration is the attraction of a cell population to the wound area with the potential to differentiate into appropriate periodontal cell types for the regeneration of the tissues of the periodontium: bone, connective tissue and cementum. Periodontal ligament and bone marrow are traditionally considered to host cell populations that retain this capacity¹⁰⁷-109. Use of PDGF and BMP-2 released in a spatial context for periodontal regeneration has the potential to control growth and differentiation of periodontal progenitors to regenerate lost structures. Regeneration of periodontal tissues can only occur once the disease-associated inflammation has been addressed, which is usually done by removal/disruption of the dental biofilm that initiated the process. Using the LPS-induced bone loss model after 4 weeks of bone loss, BMP2 and PDGF will be used in studies to determine optimal release kinetics for periodontal regeneration.

### In Vitro Determination of Biological Activity of Scaffold-Delivered Growth Factors.

The BMP-2 and PDGF release profiles from the scaffolds will be determined using an ELISA kit. The bioactivity of BMP-2 released from the scaffolds will be tested using a standard test method (ASTM F2131-02), employing pre-osteoblasts, W-20 mouse stromal cell line (W-20-17; ATCC Cat# CRL-2623). Cells will be seeded on the scaffolds loaded with or without BMP-2 and incubated for up to 28 days in a humidified atmosphere of 95% air and 5% CO₂ at 37° C. At designated time points such as day 1, 3, 5, 7, 14, 21, and 28, the cells will be then lysed by using a freeze-thaw method three times. Then, dsDNA and alkaline phosphatase activity of the cells will be evaluated by a PicoGreen assay (Invitrogen) and p-nitrophenol phosphate method (Sigma), respectively. This assay has been qualified and validated based upon the International Committee on Harmonization assay validation guidelines for the assessment of the biological activity of BMP-2. The relevance of this in vitro test method to in vivo bone formation has also been studied. The measured response in the W-20 bioassay, alkaline phosphatase induction, has been correlated with the ectopic bone-forming capacity of BMP-2 in the in vivo Use Test¹¹⁹.

The bioactivity of PDGF released from the scaffolds will be determined through rat gingival fibroblast DNA synthesis as measured by [³H]thymidine incorporation¹²⁰. Gingival fibroblasts will be seeded in the cell culture well with the addition of supernatant from scaffolds with different amounts of PDGF. 2×10⁵ cpm (count per minute) [methyl-³H]thymidine will be added to each sample well. After culture for 5 days without medium change, the medium will be removed and each well will be washed three times with cold PBS. The DNA in each well will be precipitated with 5% cold trichloroacetic acid at 4° C. for 2 h, solubilized with 1% SDS solution at 55° C. for 2 h, followed by counting the radioactivity of [methyl-³H]thymidine in the solution with a scintillation counter. Active PDGF promotes the proliferation of gingival fibroblasts in a 0-100 ng/ml range. Supernatant from scaffolds without PDGF loading will be used as negative control and PDGF solution at 50 ng/ml will be used as positive control.

### In Vivo Evaluation of Growth Factor-Containing Scaffolds.

For these studies, periodontal bone loss via A. actinomycetemcomitans LPS microinjection will be established for a 4-week period in order to evaluate the ability of the scaffolds containing a combination of growth factors, namely PDGF and BMP-2, to regenerate inflammation-induced bone loss. In studies, LPS-induced bone loss will be completely stopped (analogous to clinical debridement at the time of surgery), followed by implantation of the scaffold. The ability of this scaffold to regenerate inflammation induced bone loss will be directly compared with non-growth factor containing scaffolds over a 1 to 4 week period. The growth factor loading on the scaffold is in a spatial pattern to specifically promote bone regeneration at the alveolar site using BMP-2 and periodontal ligament regeneration at the gingival site. Therefore, at the time of NanoLbL coating, ⅓ area of a scaffold will be coated with PDGF and ⅔ area of a scaffold will be coated with BMP-2, so that BMP-2 will be delivered to the alveolar site and PDGF will be delivered for the periodontal ligament regeneration.

Each rat will have only one side (hemimaxilla) used for these studies. To evaluate the ability of BMP-2 and/or PDGF to induce periodontal regeneration, several stem cell and new connective tissue/bone markers will be assessed. Outcomes also to be studied include characterization of cell populations and of the healing tissues in the regenerating area by immunohistochemistry. Stem cell markers (e.g., CD166 (SB10, ALCAM), CD49a, Stro-1, SOX-2, CD133), and bone early indicators of bone formation (e.g., osteopontin, RP59, Bone Sialoprotein (BSP)) will be evaluated. Comparisons of immunostained markers of stem cell recruitment or new bone formation will be quantitatively scored and compared to non-growth factor containing scaffolds. In addition, sections will be immunostained for human BMP-2 and human PDGF-BB) to determine the extent of scaffold-released growth factor in the periodontal tissues of implanted animals. In serial sections, biochemical staining will be performed for osteoclast activity (e.g., TRAP), osteoblast activity and collagen production (e.g., alkaline phosphatase and Picro Sirius red, respectively). Also, the bone formation rate will be evaluated with the use of vital fluorochrome labeling of calcium by IP injections of calcein and alizarin complexone.

### Types of Growth Factors.

PDGF will be delivered for periodontal ligament regeneration and BMP-2 will be delivered for alveolar bone regeneration. Both PDGF and BMP-2 have a role in stem cell recruitment. However, if the number of stem cells in the implantation zone with PDGF or BMP-2 loaded scaffolds is not higher than blank scaffolds, HGF or SCF can be loaded, which have been shown to have a potent effect on stem cell recruitment. In addition, other growth factors, such as IGF-1 and HGF-1 have shown promising effects in periodontal tissue regeneration. The delivery of multiple growth factors can be tested if PDGF and/or BMP-2 are not effective in regeneration of LPS-induced periodontal tissue loss. If periodontal ligament regeneration with PDGF delivery is satisfied, but bone regeneration is not, BMP-2 and BMP-7 can be used in combination, pursuant to the demonstrated synergetic effect of BMP-2 and -7.

### Release Amount of Growth Factor.

10 ng/day releases from each scaffold for each growth factor are targeted in these studies. However, if the effect is not significant, the release amount can be increased up to 50 ng/day.

### Spatial Control of Growth Factor Loading.

In some embodiments, BMP-2 will be loaded only at the region(s) for bone regeneration and PDGF will be loaded only at the region(s) for periodontal ligament regeneration. However, PDGF may also be favorable for bone regeneration. Thus, if needed, PDGF can be loaded on the entire surface of the scaffold to promote both bone and soft tissue regeneration and BMP-2 can be loaded at the region for bone regeneration.

### Scaffold Materials.

In some embodiments, PLGA-collagen is used as a model scaffold material. However, the same strategy will apply for many types of biomaterials. Thus, for example, if using the PLGA-collagen scaffolds is a concern due to the acidic degradation product of PLGA, pure collagen nanofibrous scaffolds (FIG. 7B) can be used as the substrate for nanoLbL coating.

Determining the Effect of Nano-Thickness Layer-by-Layer Coatings of p38 MAPK+/−ERK Inhibitors in Combination with BMP-2/PDGF from PLGA-Collagen Scaffolds on Periodontal Tissue Regeneration from LPS Induced Inflammation and Periodontal Tissue Loss

A basic premise of this invention is that the combination of inflammatory inhibitors (e.g., p38/ERK inhibitors) to control inflammation, then bioactive agent (e.g., BMP-2/PDGF) release to regenerate LPS-induced periodontal tissue loss will be superior to either scaffolds alone or containing either inflammatory inhibitors or growth factors alone.

For these studies, the LPS model will be used to maintain an inflammatory state throughout the period initially after implantation of scaffolds alone or in combination with inhibitors and/or growth factors to mimic the human situation sustained after initial periodontal therapy (scaling and root planing along with oral hygiene instructions). This rationale stems from data in human periodontal disease tissues as described herein where significantly higher levels of P-p38 and P-ERK (FIG. 6) were observed in moderate periodontitis patients compared with healthy sites or mild periodontitis patients after scaling and root planing. These data indicate that there is potentially more inflammatory cell signaling activity in disease tissues compared with healthier tissues. Alternatively, this may reflect the infiltration of immune cells that express higher levels of phosphorylated MAPK components. To study this, an inflammatory insult will be maintained to mimic these conditions in the experimental periodontitis model by microinjection of A. actinomycetemcomitans LPS once per week over the experimental period to simulate the human scenario.

Inflammatory bone loss will be established using LPS 3 times/week for 4 weeks, then scaffolds will be implanted. The LPS injections will be continued one time/week for an additional 6 weeks to mimic lower levels of inflammation. Following baseline sacrifice in the 4-week LPS group, two other times points will be used—2 and 6 weeks after scaffold implantation. These time points were chosen to determine if short term release of p38/ERK inhibitors will be able to control inflammation (as determined by histology and IHC for cytokines/MMPs) as compared with no scaffold or non-impregnated scaffold. The later time points will allow for a determination of whether bone loss can be regenerated in this ‘clinical-like’ situation.

Following the 2 and 6 week time implantation periods, rats will be euthanized, and maxillas and serum harvested from each animal. Serum will be used to ascertain systemic cytokine levels and tartrate-resistant acid phosphate (TRAP5b) levels. For these experiments, a systemic effect of localized LPS injection is not anticipated. Data from the rat model indicates only a marginal increase in serum TNFα levels, suggesting that micro-injected LPS effects appear to be extremely local. For these proposed studies, the primary outcome assessed will be μCT analysis. The use of this technique allows for a determination of alveolar bone loss in LPS-induced rat models.

In addition to μCT and IHC, multiplex bead-based assays will be used to ascertain the amount of cytokine (IL-6, TNF and IL-1) and MMP-1, -13 expression. The same maxillas used for μCT will be decalcified and prepared for IHC analysis. Quantitative analysis of IHC staining intensity will be objectively scored by the Olympus Bliss system.

### Length of the Inhibitor Delivery and the Time for the Initiation and the Length of Growth Factor Delivery.

The delivery profile planned will first allow 2 weeks of inhibitor delivery to control the inflammation first, and then start the delivery of growth factors from the 3^{rd} week to promote regeneration after the inflammation response subsides. The delivery profile is based on clinical practice, wherein patients are normally instructed to take anti-inflammation medicine for 2 weeks to control the inflammatory response. However, this time line may not be optimal for regeneration and the profile can be adjusted to obtain full regeneration of periodontal tissue. In this system, the release profile of each component can be adjusted by control the coating protocol, i.e., the thicker the coating layer, the more loading amount. Also, the higher cross linking of the coating layer, the longer release time.

PLGA-Collagen-Based Scaffold with P38 and ERK Inhibitors.

The Nano-Layer-by-layer (NanoLbL) coating technique will be used to functionalize biodegradable PLGA-collagen-based nanofibrous scaffolds. Using this technique, surface modification of a charged template occurs via electrostatic interactions. It offers a facile method to create multifunctional films with tunable release kinetics. In particular embodiments, the release of the p38 inhibitor (SB203580) and ERK inhibitor (PD98059) followed by release of the growth factors rhBMP-2 and PDGF occurs. Crosslinked PLGA-collagen nanofibrous scaffolds have been developed using an electrospinning technique. Briefly, PLGA 50:50 copolymer (MW=51.9 kDa, Mn=34 kDa and intrinsic viscosity=0.2 dL/g; Birmingham Polymers, Inc. Birmingham, Ala.) and collagen will be dissolved in hexafluoro-2-propanol in an appropriate ratio (e.g., 7 to 1 is planned but other ratios can be used). The polymer solution will be fed by syringe pump at a controlled rate through a 23 gauge blunt tipped needle. A voltage of 5-20 kV and a working distance of 10 cm will be used. The PLGA/collagen fibers will be collected on square glass sheets on top of grounded aluminum foil. The scaffolds are then crosslinked in 5% genipin solution for one hour. Collagen, having an isoelectric point of 5.5 is negatively charged at physiologic pH, and is incorporated into the scaffold to provide a charged surface upon which the nanoLbL process can proceed¹²³. The scaffolds will be sterilized using electron beam (e-beam) sterilization.

For initial studies, the polycation poly(allylamine hydrochloride) (PAH), and the polyanion poly(acrylic acid) (PAA) will be used, although the use of other polyelectrolytes can be employed to optimize biomolecule release. Polyelectrolyte solutions will be prepared in a 0.15M NaCl solution or in ultrapure water at 1 mg/mL Growth factors, rhBMP-2 or rhPDGF, will be reconstituted in 4 mM HCl with 0.1% human serum albumin content at 10 ug/mL. Deposition times for PAH, PAA, BMP2/PDGF will be one to sixty minutes, followed by rinsing in ultrapure water.

Three loading architectures will be used to incorporate rhBMP-2 or rhPDGF or other growth factors in the bottom or internal compartment of the coating. These architectures, deep (D), double-layer (DL) and superficial (SF) correspond with the position of growth factor within the lower or internal compartment of the coating. Thus, in one embodiment, the following layering schemes will be used: Deep layer: [(growth factor)₁-(PAH/PAA)₃]; Double Layer [(PAH/PAA-BMP-22)₂-(PAH/PAA)₂]; and Superficial Layer: [(PAH/PAA)₃-BMP-2]. The subscripts represent the number of layers. A barrier nano-layer of genipin-crosslinked collagen will be used to retard release of BMP-2, PDGF and other growth factors from the lower compartment of the coating. Following deposition of the barrier layer, colloidal SB203580 and/or PD98059 particles will be deposited onto the scaffold surface with multilayer architectures to control the delivery time. SB203580 and/or PD98059 will be dissolved in DMSO at 0.1-5 ug/mL. Poly(styrene sulfonate) (PSS) is used as a counterion for PAH. The following architectures may be used (PAH/inhibitor)/(PAH/PSS)₂, (PAH/inhibitor)₂/(PAH/PSS), and (PAH/inhibitor)₃. SB203580 and PD98059 are hydrophobic and have isoelectric points around 5 to 6, rendering them negative at physiologic pH¹²⁵. Therefore, colloidal aggregates of SB203580 and/or PD98059 can be made and incorporated into coating layers. For example, SB203580 and/or PD98059 will be added to PAH at 0.5-2 mg/mL and sonicated for 30 minutes to create positively charged, soluble particulate aggregates^{126 127}. The aggregates will be centrifuged at 10,000 rpm for five minutes, rinsed with ultrapure water and resuspended in either 0.15M NaCl or ultrapure water. A deposition time of ten minutes, followed by rinsing in ultrapure water will be used for adsorption of the colloidal aggregates. A deposition time of 1-thirty minutes will be used for each layer, with ethanol rinsing followed by drying in N₂ between depositions of layers. The surface modified scaffolds will be placed in cell culture plates for in vitro release testing. At predetermined intervals, 100 uL samples will be removed and replaced with fresh PBS. Samples will be kept at −20° C. until needed for analysis. The release profiles of PD98059 and SB203580 will be determined using UV-vis spectroscopy. The amount of PD98059 and SB203580 will be quantified based on a standard concentration curve at the peak wavelengths of 260 nm and 315 nm, respectively. The bioactivity of the inhibitors will be tested with rat macrophage cell culture in the presence or absence of LPS (100 ng/ml) for 24 h. After the exposure, culture medium will be collected for ELISA to quantify IL-6 or MMP-1. The release of BMP-2, PDGF and other growth factors will be evaluated using ELISA. The bioactivity of released rhBMP-2 will be evaluated with pre-osteoblasts (W-20 mouse stromal cell line, W-20-17; ATCC Cat# CRL-2623) under ASTM F2131-02. The bioactivity of released PDGF will be examined with rat gingival fibroblast DNA synthesis as measured by [³H]thymidine incorporation. The morphology, hydrophilicity and surface charge conditions of each layer will be monitored with high resolution SEM, contact angle, XPS, quartz crystal microbalance (QCM) and zeta-potential.

### Lipopolysaccharide Preparation.

LPS from A. actinomycetemcomitans is extracted from strain Y4 (serotype B) by the hot phenol-water method as described¹²⁸. The A. actinomycetemcomitans LPS used in preliminary studies for this application contained <0.001% nucleic acid by spectrophotometry, and ca. 0.7% protein by BCA protein assay. The absence of protein in A. actinomycetemcomitans LPS preparations will be confirmed by polyacrylamide gel electrophoresis of extract samples and subsequent staining with silver nitrate and Comassie blue.

### Cytokine ELISAs.

Rat macrophage cells will be plated at 5×10⁴ cell per well in 24-well dishes with PLGA-collagen-based scaffold containing ERK (PD98059) and/or p38 MAP (SB203580) inhibitors with controls (scaffolds with no inhibitors) for 48-72 hrs then stimulated with A. actinomycetemcomitans LPS (1-5 μg/ml). Cell culture supernatants will be harvested 24 and 48 hr post stimulation. IL-6 and TNFα (and in some cases IL-10) will be measured by ELISA per manufacturer's instructions (R&D Systems). Media will be changed every 24 hours to eliminate the issues with inhibitor accumulation.

### Real Time PCR Analysis.

Total RNA will be isolated from treated cells as described above using TRIZOL (Invitrogen) according to the manufacturer's instructions and quantitated at OD₂₆₀ using a Beckman DU-600 spectrophotometer. RT-PCR will be used to analyze mRNA expression as described previously⁸⁷. Real time PCR will be performed as recently described for MMP-13 expression using Applied Biosystems Primers on Demand™⁸⁵. Briefly, cDNA is synthesized by a reverse transcription (RT) kit (Applied Biosystems) using 300 ng total RNA in a 15 uL reaction. Each reaction contained 1.5 μL, of RT-buffer, 3.3 μL of 25 mM MgCl₂, 3 μL of 10 mM dNTP, 0.75 μL of oligo dT, 0.3 μL of RNAse inhibitor, 0.37 μL of Multiscribe reverse transcriptase, and 3.77 μL of DNAse/RNAse free H₂O. Two μL of the RT reaction product will be used in a 20 μL total volume PCR reaction mix. This includes nuclease-free water, TaqMan universal PCR master mix and TaqMan gene expression assays (Applied Biosystems) for murine IL-6, TNF-α and 18S ribosomal RNA. Gene expression assays include a pair of unlabeled PCR primers and FAM-labeled internal probe; all pre-designed for detection and quantitation of gene-specific cDNA sequences. Optimized thermocycling parameters for each gene will be used. TaqMan real-time PCR will be performed on a OneStepPlus thermocycler (Applied Biosystems) and quantitated from a standard curve. Cytokine mRNA quantity in each sample is subsequently normalized to the quantity of 18S mRNA and expressed as fold change over unstimulated control.

### LPS-Induced Experimental Periodontitis Model.

A. actinomycetemcomitans LPS (10 μg/2 μl) will be microinjected into the palatal gingiva of rats using a 33 gauge needle and Hamilton syringe^{38,39,104}. Animals will be sedated using ketamine during the procedure. LPS injections will be performed 3 times/week over indicated time frames as described.

### In Vivo Bone Formation Measurements.

To follow the time course of bone formation, the rats will receive IP injections of the following fluorochrome labels: calcein (7 mg/kg, Sigma Chem. Co.) at the moment of implantation of the nanostructured scaffold (baseline) and 24 h before the sacrifice, whereas alizarin complexone (20 mg/kg, Sigma Chem. Co.) will be injected 14 days after the implantation. These fluorochromes will bind to calcium at the mineralization front and allow the observation of new bone formation by fluorescence microscopy, as previously described^{129 130}. Briefly, after harvesting, the specimens will be fixed in 10% neutral buffered formalin for 24 h at 4° C. and then transferred to 70% ethanol. Undecalcified 5 μm sections will be stained with Von Kossa/toluidine blue (2 sections/animal) according to previously described protocols¹³¹, whereas other semi-serial sections (2 sections/animal) will be visualized under a fluorescence microscope (Nikon 80i) with green (calcein, excitation 494 nm) and red filters (alizarin complexone, excitation 530-580 nm). Mineral apposition rate in each experimental group at the end of the 4-week period will be determined by the distance between the fluorochrome labels divided by the number of days between the injections¹³².

### MicroCT Analysis.

Anatomic sections will be performed to include the 3 maxillary molars, as well as surrounding osseous and soft tissues. Samples are placed in fixative (10% formalin) and scanned using a high-resolution μCT. The scanning protocol was set to obtain 18 μm resolution, and initial data reconstruction will be performed using software (GE Medical Systems, UK). Data is then exported into software for further calculation (Analyze, Analyze Direct Inc., MN). First, three-dimensional reconstruction of samples will be performed for visualization. Volumes of interest (VOI) are obtained by using digital cropping: the total field of view was first selected to include all buccal and lingual tissues two millimeters away from the highest contour of crowns. Images are reduced using a plan perpendicular to the highest contour of the first maxillary molar mesially as well as the highest contour of the 3^{rd} maxillary molar distally. Threshold will be performed to eliminate soft tissues, followed by segmentation and volume extraction. A calculation algorithm will be executed to obtain total volume. All measurements will be performed by the same trained examiners and repeated at two separate time intervals. Mean volume analyses will be compared in implanted material with different inhibitors +/−growth factors.

### Multiplex Bead-Based Assays.

Total protein will be extracted from periodontal soft tissue samples using the Bio-flex Cell lysis kit (Bio-Rad Lab) according to the manufacturer's instructions. Briefly, immediately upon collection, samples will be rinsed in wash buffer and lysed on ice in 500 μL of lysing buffer using a tissue grinder. After a 30-second sonication, samples will be centrifuged at 4,500 g for 4 minutes at 4° C. and stored at −80° C. until ready to be used. Protein concentration will be determined by the Lowry method (Bio-Rad DC protein assay), so that samples can be diluted to a standardized concentration previous to the assay (required concentration range is 200-900 ng/μL). Multiplex bead-based assays are designed in a capture sandwich immunoassay format that uses antibodies covalently coupled to color-coded 5.6 μm polystyrene beads and also biotinylated antibodies. Briefly, the procedure will be performed as follows: the 96-well filter plate will be pre-wetted with 100 μL of assay buffer, then 50 μL of the multiplex mixture of bead-conjugated antibodies will be added to each well and subsequently removed by vacuum filtration. 50 μL of standards or samples will be added to the wells and incubated for 30 min at room temperature with agitation at 300 rpm. 25 μL of biotinylated detection antibodies will be added to each well and incubated for 30 min at room temperature (300 rpm), followed by 50 μL of streptavidin-phycoerythrin (incubation at room temperature for 10 min/300 rpm). Finally, 125 μL of the assay buffer will be added to each well and the plate will be read in the Bio-Plex system and quantitated using Bio-Plex Manager software (Bio-Rad Lab) according to the manufacturer's instructions.

### TRAP Staining and Immunohistological Staining.

Following μCT analysis, mouse maxillas will be decalcified and embedded for histological preparation. Tartrate-resistant acid phosphate (TRAP) staining will be conducted as described using a leukocyte acid phosphatase kit (Sigma), at 37° C. for 20 min in a moist chamber, before counterstaining with hematoxylin. TRAP⁺ cells will be enumerated using a Nikon TS100-F microscope attached to a Nikon Evolution MP 5.1 Mega-pixel Color Cooled CCD Digital Camera. Digitally photographed TRAP⁺ cells will be counted in the whole field from sections that represent the same approximate location in the specimen. For immunohistological staining, formalin-fixed, paraffin-embedded tissue sections will be deparaffinized and rehydrated through graded ethanol solutions. Once hydrated, sections will be heated at 96° C. in Dako Target Retrieval Solution. The sections are then washed in TBS and incubated for 10 minutes with Protein Block Serum Free (Dako). The primary antibodies to be used are as described above. Sections will be incubated with the appropriate biotinylated secondary antibody for 30 minutes followed by streptavidin-biotin complex-alkaline phosphatase for an additional 30 minutes. Staining of sections is developed using a New Fuchsin substrate kit, counterstained with Mayer's hematoxylin, and mounted with Aquamount mounting medium. Primary and secondary antibodies will be diluted in Dako Antibody Diluent. Negative-staining control experiments are performed either by omitting the primary antibody or by using a control isotype-matched antibody.

### Serum Measurements of Bone Loss.

For serum measurements of bone loss induced by LPS, a rat TRAP5b kit will be used to measure serum levels of TRAP activity. TRAP5b has been shown to accurately reflect osteoclastic activity and activation¹³³. The RatTRAP™ (Immunodiagnostic Systems, Ltd.) test will be used, which is a solid-phase immunofixed enzyme activity assay for the determination of osteoclast-derived tartrate-resistant acid phosphatase form 5b (TRAP 5b) in rat serum samples. Differences between LPS-injected implanted materials with different inhibitors +/−growth factors will be determined.

### Statistical Analysis.

Pairwise comparisons between experimental groups will be performed using the student t-test with Welch's correction for unequal variances or one-way ANOVA analysis where indicated. Significance level will be set to 5%. All calculations will be performed using Prism 4 software (GraphPad, Inc.).

These studies provide a means of determining the ability of nano-layered scaffolds to control local periodontal inflammation and then promote regeneration. We propose to use cell signaling inhibitors for the p38 and ERK pathways to attenuate periodontal inflammation, then once inflammation is under control, the scaffolds will release growth factors (PDGF/BMP-2) in a spatially correct manner to promote periodontal soft tissue regeneration and bone formation.

### Example II

### Diabetic Ulcers

Diabetic ulcers are the most common foot injuries leading to lower extremity amputation. They are a major predictor of limb loss in diabetics, with a rate of 6.5 amputations per 1000 person-years, which about 10 times more than non-diabetics. They are also an accelerator of mortality for diabetic patients, with an annual incidence per diabetic patient of 2.4% to 5.6%, a prevalence of 4.4% to 7.7% and a lifetime incidence of approx. 15%

Diabetic patients with ulcers typically have reduced polymorphonuclear (PMN) leukocyte infection, reduced leukocyte chemotaxis, depressed phagocytic activity, reduced intracellular bactericidal activity and no apparent effect on the immune system.

There are several treatment options: 1) increase in blood supply (angioplasty, stent insertion, atherectomy, laser recanalization) to wound/ulcer area; 2) debridement (Necrotic tissue removal to enhance healing); 3) pressure relief (mechanical therapy, such as total contact casting); 4) infection/bioburden control (chronic wounds are known to exist along a bacterial continuum which ranges from contamination to infection); 5) moist wound healing (topical applications); 6) physical modalities (Negative pressure wound therapy-Wound Vac, electrical stimulation, magnetic therapy); 7) wound environment manipulators (PROMOGRAN) and oxygen therapy (hyperbaric oxygen, topical oxygen); and 8) active methods of healing, such as stimulation of more rapid wound healing by accelerated angiogenesis, stimulation of growth factor (GF; e.g., REGRANEX (PDGF-B)/platelet releasates; epidermal growth factor (EGF), fibroblast growth factor (bFGF), granulocyte macrophage colony stimulating factor (GM-CSF), keratinocyte growth factor-2 (KGF-2), transforming growth factor beta (TGF-β)) release, introduction of wound matrix for cellular ingrowth and/or production of required proteins/GFs; platelet-derived growth factor (e.g., Becaplermin) application and living human dermal substitutes (e.g., APLIGRAF, DERMAGRAFT).

Despite use of optimal therapy, diabetic ulcers require an average of 4-6 months of treatment to heal and many patients cannot tolerate the requirements of treatment for 4-6 months or more. The cost in terms of lost productivity, impact on work, exercise and lifestyle is high.

The barriers to diabetic wound healing are the incapable control of local environment. The microenvironment in the ulcer is very complicated and involves formation of a biofilm that is resistant to antibiotic treatment and production of bacterial toxins (e.g., endotoxins, such as lipopolysaccharide, (LPS)).

LPS can further cause inflammation, e.g., via IL-1, IL-6 and/or TNF-alpha up-regulation. It can also cause up-regulation of matrix metalloproteinases (MMPs), which break down the extracellular matrix (ECM) and down-expression of growth factors for diabetic ulcers, such as TGF-beta, PDGF, etc.

As the pathology of diabetic ulcer is similar to that of periodontal disease, the compositions and methods of this invention for treatment of periodontal disease can also be employed for the treatment of diabetic ulcers, with logistical and regimental modifications appropriate to the different environments, as would be apparent to one of ordinary skill in the art.

Specifically, attenuating LPS-elicited inflammatory responses is needed to decrease inflammation permitting subsequent regenerative therapies for both periodontal diseases and diabetic ulcers.

Recent studies indicate that p38 mitogen-activated protein kinase (MAPK) is a major signaling pathway needed to mediate LPS-induced local tissue loss. The tissue preservation observed with p38 inhibitors was due to the decrease in the production of inflammatory cytokines at the post-transcriptional level leading to suppression of tissue regeneration.

The (ERK) MAPK pathway is also needed, in addition to p38 MAPK, for LPS-stimulated matrix metalloproteinases (MMPs) and other proinflammatory cytokines in mononuclear cells.

Biodegradable scaffolds with nanothickness layer-by-layer (nanoLbL) drug coating are effective in the delivery of 1) antibiotics and/or antimicrobials to treat infection, 2) anti-inflammatory agents such as p38 and ERK inhibitors that can attenuate LPS-elicited inflammatory responses; and 3) different growth factors that can promote cell migration, growth, proliferation and/or differentiation to regenerate the lost tissue.

Owing to rapid clearance in vivo and the inability to maintain a therapeutic concentration of growth factors, a local long-term delivery strategy would be ideal for a growth factor-based therapy.

Without control of infection and/or inflammation, regeneration will not be successful. With anti-inflammation treatment, the regeneration process may be compromised since signaling through p38 and ERK pathways is required for growth factor induced regeneration. Therefore, an ideal delivery scheme would first promote inflammation resolution with short-term delivery of p38 and/or ERK inhibitors and then growth factor delivery for regeneration. A degradable scaffold with nanoLbL coatings of different agents at different layers allows sequentially controlled delivery of small molecules inhibitors and growth factors from a single scaffold. Thus, in some embodiments, the scaffolds of this invention can be used to sequentially deliver p38 MAPK inhibitor +/−ERK inhibitor in combination with subsequent release of growth factor(s) for tissue regeneration to temporally control inflammation and promote further regeneration.

Using the approach described herein, surface modification of a charged template occurs via electrostatic interactions. This offers a facile method to create multifunctional films with tunable release kinetics.

This invention provides a compartmentalization technique that allows for multimolecule release. One example is: release of the p38 inhibitor (SB203580) and ERK inhibitor (PD98059) followed by release of the growth factors rhBMP-2 and PDGF.

Crosslinked PLGA-collagen nanofibrous scaffolds have been developed using an electrospinning technique. In one example, PLGA 50:50 copolymer and collagen were dissolved in hexafluoro-2-propanol in an appropriate ratio. The polymer solution was fed by syringe pump at a controlled rate through a 23 gauge blunt tipped needle. A voltage of 5-20 kV and a working distance of 10 cm were used. The PLGA/collagen fibers were collected on square glass sheets on top of grounded aluminum foil. The scaffolds were then crosslinked in 5% genipin solution for one hour. Collagen, having an isoelectric point of 5.5 is negatively charged at physiologic pH, and is incorporated into the scaffold to provide a charged surface upon which the nanoLbL process can proceed. The scaffolds are sterilized using electron beam (e-beam) sterilization.

In one example, polycation poly(allylamine hydrochloride) (PAH), and the polyanion poly(acrylic acid) (PAA) were used, although other polyelectrolytes can be used to optimize biomolecule release. Polyelectrolyte solutions were prepared in 0.15M NaCl solution or in ultrapure water at 1 mg/mL. Growth factors, rhBMP-2 or rhPDGF were reconstituted in 4 mM HCl with 0.1% human serum albumin content at 10 ug/mL. Deposition times for PAH, PAA, BMP2/PDGF were one to sixty minutes, followed by rinsing in ultrapure water. Three loading architectures were used to incorporate rhBMP-2 or rhPDGF or other growth factors in the inner (e.g., bottom or lower) compartment of the coating.

These architectures, deep (D), double-layer (DL) and superficial (SF), correspond with the position of growth factor within the inner compartment of the coating. Thus, the following layering schemes were used: deep layer [(growth factor)₁-(PAH/PAA)₃]; double Layer [(PAH/PAA-BMP2)₂-(PAH/PAA)₂]; and superficial layer [(PAH/PAA)₃-BMP2]. The subscripts represent the number of layers.

A barrier nano-layer of genipin-crosslinked collagen was used to retard release of BMP2, PDGF and other growth factors from the inner compartment of the coating.

Following deposition of the barrier layer, colloidal SB203580 and/or PD98059 particles were deposited onto the scaffold surface with multilayer architectures to control the delivery time.

SB203580 and/or PD98059 were dissolved in DMSO at 0.1-5 ug/mL Poly(styrene sulfonate) (PSS) was used as a counterion for PAH. The following architectures were used (PAH/inhibitor)/(PAH/PSS)₂, (PAH/inhibitor)₂/(PAH/PSS), and (PAH/inhibitor)₃.

SB203580 and PD98059 are hydrophobic and have isoelectric points around 5 to 6, rendering them negative at physiologic pH. Therefore, colloidal aggregates of SB203580 and/or PD98059 can be made and incorporated into coating layers. For example, SB203580 and/or PD98059 were added to PAH at 0.5-2 mg/mL and sonicated for 30 minutes to create positively charged, soluble particulate aggregates. The aggregates were centrifuged at 10,000 rpm for five minutes, rinsed with ultrapure water, and resuspended in either 0.15M NaCl or ultrapure water. A deposition time of ten minutes, followed by rinsing in ultrapure water was used for adsorption of the colloidal aggregates. A deposition time of 1-thirty minutes was used for each layer, and ethanol rinsing followed by drying in N2 occurred between depositions of layers.

The surface modified scaffolds were placed in cell culture plates for in vitro release testing. At predetermined intervals, 100 uL samples were removed and replaced with fresh PBS. Samples were kept at −20° C. until needed for analysis.

The release profiles of PD98059 and SB203580 were determined using UV-vis spectroscopy. The amount of PD98059 and SB203580 was quantified based on a standard concentration curve at the peak wavelengths of 260 nm and 315 nm, respectively.

The bioactivity of the inhibitors was tested with rat macrophage cell culture in the presence or absence of LPS (100 ng/ml) for 24 h. After the exposure, culture medium was collected for ELISA to quantify IL-6 or MMP-1. The release of BMP-2, PDGF and other growth factors were evaluated using ELISA. The morphology, hydrophilicity, and surface charge conditions of each layer were monitored with zeta-potential. (Table 2).

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

### REFERENCES

1. Nakashima T, Kobayashi Y, Yamasaki S, Kawakami A, Eguchi K, Sasaki H, Sakai H. Protein expression and functional difference of membrane-bound and soluble receptor activator of NF-kappaB ligand: modulation of the expression by osteotropic factors and cytokines. Biochem Biophys Res Commun 2000; 275 (3):768-75.
2. Jiang Y, Mehta C K, Hsu T Y, Alsulaimani F F. Bacteria induce osteoclastogenesis via an osteoblast-independent pathway. Infect Immun 2002; 70 (6):3143-8.
3. Kikuchi T, Yoshikai Y, Miyoshi J, Katsuki M, Musikacharoen T, Mitani A, Tanaka S, Noguchi T, Matsuguchi T. Cot/Tpl2 is essential for RANKL induction by lipid A in osteoblasts. J Dent Res 2003; 82 (7):546-50.
4. Yang H, Young D W, Gusovsky F, Chow J C. Cellular events mediated by lipopolysaccharide-stimulated toll-like receptor 4. MD-2 is required for activation of mitogen-activated protein kinases and Elk-1. J Biol Chem 2000; 275 (27):20861-6.
5. Kirschning C J, Bauer S. Toll-like receptors: cellular signal transducers for exogenous molecular patterns causing immune responses. Int J Med Microbiol 2001; 291 (4):251-60.
6. Kirschning C J, Wesche H, Merrill Ayres T, Rothe M. Human toll-like receptor 2 confers responsiveness to bacterial lipopolysaccharide. J Exp Med 1998; 188 (11):2091-7.
7. Roux S, Orcel P. Bone loss. Factors that regulate osteoclast differentiation: an update. Arthritis Res 2000; 2 (6):451-456.
8. Reddi K, Wilson M, Nair S, Poole S, Henderson B. Comparison of the pro-inflammatory cytokine-stimulating activity of the surface-associated proteins of periodontopathic bacteria. J Periodontal Res 1996; 31 (2):120-30.
9. Lee H J, Kang I K, Chung C P, Choi S M. The subgingival microflora and gingival crevicular fluid cytokines in refractory periodontitis. J Clin Periodontol 1995; 22 (11):885-90.
10. Ejeil A L, Gaultier F, Igondjo-Tchen S, Senni K, Pellat B, Godeau G, Gogly B. Are cytokines linked to collagen breakdown during periodontal disease progression? J Periodontol 2003; 74 (2):196-201.
11. Gamonal J, Acevedo A, Bascones A, Jorge O, Silva A. Levels of interleukin-1 beta, -8, and -10 and RANTES in gingival crevicular fluid and cell populations in adult periodontitis patients and the effect of periodontal treatment. J Periodontol 2000; 71 (10):1535-45.
12. Geivelis M, Turner D W, Pederson E D, Lamberts B L. Measurements of interleukin-6 in gingival crevicular fluid from adults with destructive periodontal disease. J Periodontol 1993; 64 (10):980-3.
13. Gorska R, Gregorek H, Kowalski J, Laskus-Perendyk A, Syczewska M, Madalinski K. Relationship between clinical parameters and cytokine profiles in inflamed gingival tissue and serum samples from patients with chronic periodontitis. J Clin Periodontol 2003; 30 (12):1046-52.
14. Stashenko P, Jandinski J J, Fujiyoshi P, Rynar J, Socransky S S. Tissue levels of bone resorptive cytokines in periodontal disease. J Periodontol 1991; 62 (8):504-9.
15. Graves D T. The potential role of chemokines and inflammatory cytokines in periodontal disease progression. Clin Infect Dis 1999; 28 (3):482-90.
16. Graves D T, Delima A J, Assuma R, Amar S, Oates T, Cochran D. Interleukin-1 and tumor necrosis factor antagonists inhibit the progression of inflammatory cell infiltration toward alveolar bone in experimental periodontitis. J Periodontol 1998; 69 (12):1419-25.
17. Janeway C A, Jr., Medzhitov R. Innate immune recognition. Annu Rev Immunol 2002; 20:197-216.
18. Mori Y, Yoshimura A, Ukai T, Lien E, Espevik T, Hara Y. Immunohistochemical localization of Toll-like receptors 2 and 4 in gingival tissue from patients with periodontitis. Oral Microbiol Immunol 2003; 18 (1):54-8.
19. An H, Yu Y, Zhang M, Xu H, Qi R, Yan X, Liu S, Wang W, Guo Z, Guo J and others. Involvement of ERK, p38 and NF-kappaB signal transduction in regulation of TLR2, TLR4 and TLR9 gene expression induced by lipopolysaccharide in mouse dendritic cells. Immunology 2002; 106 (1):38-45.
20. Schumann R R, Pfeil D, Lamping N, Kirschning C, Scherzinger G, Schlag P, Karawajew L, Herrmann F. Lipopolysaccharide induces the rapid tyrosine phosphorylation of the mitogen-activated protein kinases erk-1 and p38 in cultured human vascular endothelial cells requiring the presence of soluble CD14. Blood 1996; 87 (7):2805-14.
21. Bainbridge B W, Darveau R P. Porphyromonas gingivalis lipopolysaccharide: an unusual pattern recognition receptor ligand for the innate host defense system. Acta Odontol Scand 2001; 59 (3):131-8.
22. Genco C A, Van Dyke T, Amar S. Animal models for Porphyromonas gingivalis-mediated periodontal disease. Trends Microbiol 1998; 6 (11):444-9.
23. Listgarten M A, Wong M Y, Lai C H. Detection of Actinobacillus actinomycetemcomitans, Porphyromonas gingivalis, and Bacteroides forsythus in an A. actinomycetemcomitans-positive patient population. J Periodontol 1995; 66 (2):158-64.
24. Hirschfeld M, Ma Y, Weis J H, Vogel S N, Weis J J. Cutting edge: repurification of lipopolysaccharide eliminates signaling through both human and murine toll-like receptor 2. J Immunol 2000; 165 (2):618-22.
25. Suda T, Kobayashi K, Jimi E, Udagawa N, Takahashi N. The molecular basis of osteoclast differentiation and activation. Novartis Found Symp 2001; 232:235-47; discussion 247-50.
26. Delima A J, Oates T, Assuma R, Schwartz Z, Cochran D, Amar S, Graves D T. Soluble antagonists to interleukin-1 (IL-1) and tumor necrosis factor (TNF) inhibits loss of tissue attachment in experimental periodontitis. J Clin Periodontol 2001; 28 (3):233-40.
27. Marshall C J. MAP kinase kinase kinase, MAP kinase kinase and MAP kinase. Curr Opin Genet Dev 1994; 4 (1):82-9.
28. Raingeaud J, Gupta S, Rogers J S, Dickens M, Han J, Ulevitch R J, Davis R J. Pro-inflammatory cytokines and environmental stress cause p38 mitogen-activated protein kinase activation by dual phosphorylation on tyrosine and threonine. J Biol Chem 1995; 270 (13):7420-6.
29. Cobb M H, Goldsmith E J. How MAP kinases are regulated. J Biol Chem 1995; 270 (25):14843-6.
30. Lee J C, Kassis S, Kumar S, Badger A, Adams J L. p38 mitogen-activated protein kinase inhibitors—mechanisms and therapeutic potentials. Pharmacol Ther 1999; 82 (2-3):389-97.
31. Lee J C, Kumar S, Griswold D E, Underwood D C, Votta B J, Adams J L. Inhibition of p38 MAP kinase as a therapeutic strategy. Immunopharmacology 2000; 47 (2-3):185-201.
32. Lee J C, Laydon J T, McDonnell P C, Gallagher T F, Kumar S, Green D, McNulty D, Blumenthal M J, Heys J R, Landvatter S W and others. A protein kinase involved in the regulation of inflammatory cytokine biosynthesis. Nature 1994; 372 (6508):739-46.
33. Raingeaud J, Whitmarsh A J, Barrett T, Derijard B, Davis R J. MKK3- and MKK6-regulated gene expression is mediated by the p38 mitogen-activated protein kinase signal transduction pathway. Mol Cell Biol 1996; 16 (3):1247-55.
34. Chae H J, Chae S W, Chin H Y, Bang B G, Cho S B, Han K S, Kim S C, Tae K C, Lee K H, Kim D E and others. The p38 mitogen-activated protein kinase pathway regulates interleukin-6 synthesis in response to tumor necrosis factor in osteoblasts. Bone 2001; 28 (1):45-53.
35. Chae H J, Kim S C, Chae S W, An N H, Kim H H, Lee Z H, Kim H R. Blockade of the p38 mitogen-activated protein kinase pathway inhibits inducible nitric oxide synthase and interleukin-6 expression in MC3T3E-1 osteoblasts. Pharmacol Res 2001; 43 (3):275-83.
36. Webb S J, McPherson J R, Pahan K, Koka S. Regulation of TNF-alpha-induced IL-6 production in MG-63 human osteoblast-like cells. J Dent Res 2002; 81 (1):17-22.
37. Kumar S, Votta B J, Rieman D J, Badger A M, Gowen M, Lee J C. IL-1- and TNF-induced bone resorption is mediated by p38 mitogen activated protein kinase. J Cell Physiol 2001; 187 (3):294-303.
38. Kirkwood K, Li F, Rogers J, Otremba J, Coatney D, Kreider J, D'Silva N, Chakravarty S, Dugar S, Higgins L and others. A p38 alpha selective mitogen-activated protein kinase inhibitor prevents periodontal bone loss. J Pharmacol Exp Ther 2007; 320 (1):56-63.
39. Rogers J E, Li F, Coatney D D, Otremba J, Kriegl J M, Protter T A, Higgins L S, Medicherla S, Kirkwood K L. A p38 mitogen-activated protein kinase inhibitor arrests active alveolar bone loss in a rat periodontitis model. J Periodontol 2007; 78 (10):1992-8.
40. Derrigo M, Cestelli A, Savettieri G, Di Liegro I. RNA-protein interactions in the control of stability and localization of messenger RNA (review). Int J Mol Med 2000; 5 (2):111-23.
41. Grzybowska E A, Wilczynska A, Siedlecki J A. Regulatory functions of 3′UTRs. Biochem Biophys Res Commun 2001; 288 (2):291-5.
42. Thalhamer T, McGrath M A, Harnett M M. MAPKs and their relevance to arthritis and inflammation. Rheumatology (Oxford) 2008; 47 (4):409-14.
43. Dong C, Davis R J, Flavell R A. MAP kinases in the immune response. Annu Rev Immunol 2002; 20:55-72.
44. Marie C, Roman-Roman S, Rawadi G. Involvement of mitogen-activated protein kinase pathways in interleukin-8 production by human monocytes and polymorphonuclear cells stimulated with lipopolysaccharide or Mycoplasma fermentans membrane lipoproteins. Infect Immun 1999; 67 (2):688-93.
45. Lai W C, Zhou M, Shankavaram U, Peng G, Wahl L M. Differential regulation of lipopolysaccharide-induced monocyte matrix metalloproteinase (MMP)-1 and MMP-9 by p38 and extracellular signal-regulated kinase 1/2 mitogen-activated protein kinases. J Immunol 2003; 170 (12):6244-9.
46. Reimann T, Buscher D, Hipskind R A, Krautwald S, Lohmann-Matthes M L, Baccarini M. Lipopolysaccharide induces activation of the Raf-1/MAP kinase pathway. A putative role for Raf-1 in the induction of the IL-1 beta and the TNF-alpha genes. J Immunol 1994; 153 (12):5740-9.
47. Borden P, Heller R A. Transcriptional control of matrix metalloproteinases and the tissue inhibitors of matrix metalloproteinases. Crit Rev Eukaryot Gene Expr 1997; 7 (1-2):159-78.
48. Pillinger M H, Marjanovic N, Kim S Y, Lee Y C, Scher J U, Roper J, Abeles A M, Izmirly P I, Axelrod M, Pillinger M Y and others. Helicobacter pylori stimulates gastric epithelial cell MMP-1 secretion via CagA-dependent and -independent ERK activation. J Biol Chem 2007; 282 (26):18722-31.
49. Williams T N, Zhang C X, Game B A, He L, Huang Y. C-reactive protein stimulates MMP-1 expression in U937 histiocytes through Fc[gamma]RII and extracellular signal-regulated kinase pathway:: an implication of CRP involvement in plaque destabilization. Arterioscler Thromb Vasc Biol 2004; 24 (1):61-6.
50. Jinnin M, Ihn H, Mimura Y, Asano Y, Yamane K, Tamaki K. Matrix metalloproteinase-1 up-regulation by hepatocyte growth factor in human dermal fibroblasts via ERK signaling pathway involves Ets1 and Fli1. Nucleic Acids Res 2005; 33 (11):3540-9.
51. Park M J, Park I C, Lee H C, Woo S H, Lee J Y, Hong Y J, Rhee C H, Lee Y S, Lee S H, Shim B S and others. Protein kinase C-alpha activation by phorbol ester induces secretion of gelatinase B/MMP-9 through ERK 1/2 pathway in capillary endothelial cells. Int J Oncol 2003; 22 (1): 137-43.
52. Sundararaj K P, Samuvel D J, Li Y, Nareika A, Slate E H, Sanders J J, Lopes-Virella M F, Huang Y. Simvastatin suppresses LPS-induced MMP-1 expression in U937 mononuclear cells by inhibiting protein isoprenylation-mediated ERK activation. J Leukoc Biol 2008; 84 (4): 1120-9.
53. Pelletier J P, Fernandes J C, Brunet J, Moldovan F, Schrier D, Flory C, Martel-Pelletier J. In vivo selective inhibition of mitogen-activated protein kinase kinase 1/2 in rabbit experimental osteoarthritis is associated with a reduction in the development of structural changes. Arthritis Rheum 2003; 48 (6):1582-93.
54. Wozney J M. The bone morphogenetic protein family: multifunctional cellular regulators in the embryo and adult. Eur J Oral Sci 1998; 106 Suppl 1:160-6.
55. Thesleff I, Vaahtokari A, Vainio S, Jowett A. Molecular mechanisms of cell and tissue interactions during early tooth development. Anat Rec 1996; 245 (2):151-61.
56. Meraw S J, Reeve C M, Lohse C M, Sioussat T M. Treatment of peri-implant defects with combination growth factor cement. J Periodontol 2000; 71 (1):8-13.
57. Hollinger J O, Hart C E, Hirsch S N, Lynch S, Friedlaender G E. Recombinant human platelet-derived growth factor: biology and clinical applications. J Bone Joint Surg Am 2008; 90 Suppl 1:48-54.
58. King G N, Cochran D L. Factors that modulate the effects of bone morphogenetic protein-induced periodontal regeneration: a critical review. J Periodontol 2002; 73 (8):925-36.
59. King G N, King N, Cruchley A T, Wozney J M, Hughes F J. Recombinant human bone morphogenetic protein-2 promotes wound healing in rat periodontal fenestration defects. J Dent Res 1997; 76 (8):1460-70.
60. Zhang Y, Wang Y, Shi B, Cheng X, A platelet-derived growth factor releasing chitosan/coral composite scaffold for periodontal tissue engineering. Biomaterials 2007; 28 (8):1515-22.
61. Chen F M, Wu Z F, Sun H H, Wu H, Xin S N, Wang Q T, Dong G Y, Ma Z W, Huang S, Zhang Y J and others. Release of bioactive BMP from dextran-derived microspheres: a novel delivery concept. Int J Pharm 2006; 307 (1):23-32.
62. Jovanovic S A, Hunt D R, Bernard G W, Spiekermann H, Wozney J M, Wikesjo U M. Bone reconstruction following implantation of rhBMP-2 and guided bone regeneration in canine alveolar ridge defects. Clin Oral Implants Res 2007; 18 (2):224-30.
63. Park J B, Matsuura M, Han K Y, Norderyd O, Lin W L, Genco R J, Cho M I. Periodontal regeneration in class III furcation defects of beagle dogs using guided tissue regenerative therapy with platelet-derived growth factor. J Periodontol 1995; 66 (6):462-77.
64. Fujita T, Shiba H, Van Dyke T E, Kurihara H. Differential effects of growth factors and cytokines on the synthesis of SPARC, DNA, fibronectin and alkaline phosphatase activity in human periodontal ligament cells. Cell Biol Int 2004; 28 (4):281-6.
65. Pan S X, Li Y, Feng H L, Bai W, Gu Y Y. In vitro aging of mineralized collagen-based composite as guided tissue regeneration membrane. Materials Science and Engineering: C 2006; 26 (4):724-729.
66. Owen G R, Jackson J, Chehroudi B, Burt H, Brunette D M. A PLGA membrane controlling cell behavior for promoting tissue regeneration. Biomaterials 2005; 26 (35):7447-56.
67. Kuo T F, Huang A T, Chang H H, Lin F H, Chen S T, Chen R S, Chou C H, Lin H C, Chiang H, Chen M H. Regeneration of dentin-pulp complex with cementum and periodontal ligament formation using dental bud cells in gelatin-chondroitin-hyaluronan tri-copolymer scaffold in swine. J Biomed Mater Res A 2008; 86 (4):1062-8.
68. Taba M, Jr., Jin Q, Sugai J V, Giannobile W V. Current concepts in periodontal bioengineering. Orthod Craniofac Res 2005; 8 (4):292-302.
69. Zhang Y-F, Cheng X-R, Yun C, Bin S, Chen X-H, Xu D-X, Jin K. Three-dimensional Nanohydroxyapatite/Chitosan Scaffolds as Potential Tissue Engineered Periodontal Tissue. J Biomater Appl 2007; 21 (4):333-349.
70. Hong H, Wei J, Liu C. Development of asymmetric gradational-changed porous chitosan membrane for guided periodontal tissue regeneration. Composites. Part B, Engineering 2007; 38 (3):311-316.
71. Kim K H, Jeong L, Park H N, Shin S Y, Park W H, Lee S C, Kim T I, Park Y J, Seol Y J, Lee. Y M and others. Biological efficacy of silk fibroin nanofiber membranes for guided bone regeneration. J Biotechnol 2005; 120 (3):327-39.
72. Zhang Y, Cheng X, Wang J, Wang Y, Shi B, Huang C, Yang X, Liu T. Novel chitosan/collagen scaffold containing transforming growth factor-beta1 DNA for periodontal tissue engineering. Biochem Biophys Res Commun 2006; 344 (1):362-9.
73. Cooke J W, Sarment D P, Whitesman L A, Miller S E, Jin Q, Lynch S E, Giannobile W V. Effect of rhPDGF-BB delivery on mediators of periodontal wound repair. Tissue Eng 2006; 12 (6):1441-50.
74. Chen F M, Zhao Y M, Wu H, Deng Z H, Wang Q T, Zhou W, Liu Q, Dong G Y, Li K, Wu Z F and others. Enhancement of periodontal tissue regeneration by locally controlled delivery of insulin-like growth factor-I from dextran-co-gelatin microspheres. J Control Release 2006; 114 (2):209-22.
75. Zhang C C, Lodish H F. Murine hematopoietic stem cells change their surface phenotype during ex vivo expansion. Blood 2005; 105 (11):4314-20.
76. Banfi A, Muraglia A, Dozin B, Mastrogiacomo M, Cancedda R, Quarto R. Proliferation kinetics and differentiation potential of ex vivo expanded human bone marrow stromal cells: Implications for their use in cell therapy. Exp Hematol 2000; 28 (6):707-15.
77. Muraglia A, Cancedda R, Quarto R. Clonal mesenchymal progenitors from human bone marrow differentiate in vitro according to a hierarchical model. J Cell Sci 2000; 113 (Pt 7):1161-6.
78. Neuss S, Becher E, Woltje M, Tietze L, Jahnen-Dechent W. Functional expression of HGF and HGF receptor/c-met in adult human mesenchymal stem cells suggests a role in cell mobilization, tissue repair, and wound healing. Stem Cells 2004; 22 (3):405-14.
79. Zhong R, Law P, Wong D, Merzouk A, Salari H, Ball E D. Small peptide analogs to stromal derived factor-1 enhance chemotactic migration of human and mouse hematopoietic cells. Exp Hematol 2004; 32 (5):470-5.
80. Fiedler J, Roderer G, Gunther K P, Brenner R E. BMP-2, BMP-4, and PDGF-bb stimulate chemotactic migration of primary human mesenchymal progenitor cells. J Cell Biochem 2002; 87 (3):305-12.
81. Cianciolo G, Stefoni S, Zanchelli F, Iannelli S, Coli L, Borgnino L C, De Sanctis L B, Stefoni V, De Pascalis A, Isola E and others. PDGF-AB release during and after haemodialysis procedure. Nephrol Dial Transplant 1999; 14 (10):2413-9.
82. Rossa C, Jr., Liu M, Bronson P, Kirkwood K L. Transcriptional activation of MMP-13 by periodontal pathogenic LPS requires p38 MAP kinase. J Endotoxin Res 2007; 13 (2):85-93.
85. Rossa C, Ehmann K, Liu M, Patil C, Kirkwood K L. MKK3/6-p38 MAPK signaling is required for IL-1beta and TNF-alpha-induced RANKL expression in bone marrow stromal cells. J Interferon Cytokine Res 2006; 26 (10):719-29.
86. Rossa C, Jr., Liu M, Kirkwood K L. A dominant function of p38 mitogen-activated protein kinase signaling in receptor activator of nuclear factor-kappaB ligand expression and osteoclastogenesis induction by Aggregatibacter actinomycetemcomitans and Escherichia coli lipopolysaccharide. J Periodontal Res 2008; 43 (2):201-11.
87. Rossa C, Jr., Liu M, Patil C, Kirkwood K L. MKK3/6-p38 MAPK negatively regulates murine MMP-13 gene expression induced by IL-1beta and TNF-alpha in immortalized periodontal ligament fibroblasts. Matrix Biol 2005; 24 (7):478-88.
88. Patil C, Rossa C, Jr., Kirkwood K L. Actinobacillus actinomycetemcomitans lipopolysaccharide induces interleukin-6 expression through multiple mitogen-activated protein kinase pathways in periodontal ligament fibroblasts. Oral Microbiol Immunol 2006; 21 (6):392-8.
98. Patil C, Zhu X, Rossa C, Jr., Kim Y J, Kirkwood K L. p38 MAPK regulates IL-1beta induced IL-6 expression through mRNA stability in osteoblasts. Immunol Invest 2004; 33 (2):213-33.
99. Patil C S, Liu M, Zhao W, Coatney D D, Li F, VanTubergen E A, D'Silva N J, Kirkwood K L. Targeting mRNA stability arrests inflammatory bone loss. Mol Ther 2008; 16 (10):1657-64.
100. Klausen B. Microbiological and immunological aspects of experimental periodontal disease in rats: a review article. J Periodontol 1991; 62 (1):59-73.
101. Garlet G P, Avila-Campos M J, Milanezi C M, Ferreira B R, Silva J S. Actinobacillus actinomycetemcomitans-induced periodontal disease in mice: patterns of cytokine, chemokine, and chemokine receptor expression and leukocyte migration. Microbes Infect 2005; 7 (4): 738-47.
102. Garlet G P, Cardoso C R, Silva T A, Ferreira B R, Avila-Campos M J, Cunha F Q, Silva J S. Cytokine pattern determines the progression of experimental periodontal disease induced by Actinobacillus actinomycetemcomitans through the modulation of MMPs, RANKL, and their physiological inhibitors. Oral Microbiol Immunol 2006; 21 (1):12-20.
103. Dumitrescu A L, Abd-El-Aleem S, Morales-Aza B, Donaldson L F. A model of periodontitis in the rat: effect of lipopolysaccharide on bone resorption, osteoclast activity, and local peptidergic innervation. J Clin Periodontol 2004; 31 (8):596-603.
104. Rogers J E, Li F, Coatney D D, Rossa C, Bronson P, Krieder J M, Giannobile W V, Kirkwood K L. Actinobacillus actinomycetemcomitans lipopolysaccharide-mediated experimental bone loss model for aggressive periodontitis. J Periodontol 2007; 78 (3):550-8.
105. Xiao Y Q, Malcolm K, Worthen G S, Gardai S, Schiemann W P, Fadok V A, Bratton D L, Henson P M. Cross-talk between ERK and p38 MAPK mediates selective suppression of pro-inflammatory cytokines by transforming growth factor-beta. J Biol Chem 2002; 277 (17):14884-93.
106. Gazel A, Nijhawan R I, Walsh R, Blumenberg M. Transcriptional profiling defines the roles of ERK and p38 kinases in epidermal keratinocytes. J Cell Physiol 2008; 215 (2):292-308.
107. Polson A M, Caton J. Factors influencing periodontal repair and regeneration. J Periodontol 1982; 53 (10):617-25.
108. Karring T, Nyman S, Lindhe J. Healing following implantation of periodontitis affected roots into bone tissue. J Clin Periodontol 1980; 7 (2):96-105.
109. Nyman S, Karring T, Lindhe J, Planten S. Healing following implantation of periodontitis-affected roots into gingival connective tissue. J Clin Periodontol 1980; 7 (5):394-401.
119. www.astm.org/Standards/F2131.htm.
120. Wei G, Jin Q, Giannobile W V, Ma P X. Nano-fibrous scaffold for controlled delivery of recombinant human PDGF-BB. J Control Release 2006; 112 (1):103-10.
123. Barbani N, Lazzeri L, Cristallini C, Cascone M G, Polacco G, Pizzirani G. Bioartificial materials based on blends of collagen and poly(acrylic acid). Journal of Applied Polymer Science 1999; 72:971-976.
125. Hedges J C, Yamboliev I A, Ngo M, Horowitz B, Adam L P, Gerthoffer W T. p38 mitogen-activated protein kinase expression and activation in smooth muscle. Am J Physiol 1998; 275 (2 Pt 1):C527-34.
126. Stewart S S, Roldan J E, Lvov Y M, Mills D K. Layer-by-Layer adsorption of biocompatible polyelectrolytes onto dexamethasone aggregates. Conf Proc IEEE Eng Med Biol Soc 2006; 1:1474-7.
127. Agarwal A, Lvov Y, Sawant R, Torchilin V. Stable nanocolloids of poorly soluble drugs with high drug content prepared using the combination of sonication and layer-by-layer technology. J Control Release 2008; 128 (3):255-60.
128. Wilson M E, Hamilton R G. Immunoglobulin G subclass response of localized juvenile periodontitis patients to Actinobacillus actinomycetemcomitans Y4 lipopolysaccharide. Infect Immun 1992; 60 (5):1806-12.
129. Forwood M R, Owan I, Takano Y, Turner C H. Increased bone formation in rat tibiae after a single short period of dynamic loading in vivo. Am J Physiol 1996; 270 (3 Pt 1):E419-23.
130. Pautke C, Vogt S, Tischer T, Wexel G, Deppe H, Milz S, Schieker M, Kolk A. Polychrome labeling of bone with seven different fluorochromes: enhancing fluorochrome discrimination by spectral image analysis. Bone 2005; 37 (4):441-5.
131. Erben R G, Kohn B, Rambeck W A, Zucker H. Histomorphometric analysis of the rat proximal tibial metaphysis by “linear scanning”. Scanning Microsc 1990; 4 (3):625-38; discussion 638-40.
132. Daci E, Verstuyf A, Moermans K, Bouillon R, Carmeliet G. Bone resorption induced by 1 alpha,25 dihydroxyvitamin D(3) in vivo is not altered by inactivation of the plasminogen activator inhibitor 1. Bone 2000; 27 (1):97-102.
133. Leeming D J, Koizumi M, Byrjalsen I, Li B, Qvist P, Tanko L B. The relative use of eight collagenous and noncollagenous markers for diagnosis of skeletal metastases in breast, prostate, or lung cancer patients. Cancer Epidemiol Biomarkers Prev 2006; 15 (1):32-8.

TABLE 1
Target genes regulated by p38 and ERK MAPK pathways

| | Genes down regulated | Transcription factors | |
|---|---|---|---|
| | by inhibiting the | activated by the | |
| MAP Kinase pathways | pathways | pathways | Supporting references |
| p38 | CXCL-2, TNF-α, IL-1β, | c-Fos, SRY, N-Myc, | 32, 33, 45, 47-62 |
| | IL-6, IL-8, MMP-1, MMP- | Foxo-1, ATF-1, ETS-1, | |
| | 3, MMP-10, MMP-13, | Elk-1, p53, STAT1, C- | |
| | RANKL, TLR2, BMP-2 | EBP-β | |
| ERK 1/2 | TNF-α, IL-6, IL-8, MMP- | Elk-1, SAP1, HLH2, | |
| | 1, MMP-9, MMP-13 | Foxo-1, ATF-1, C/EBP-β | |

TABLE 2

| | ζ-potential |
|---|---|
| Membrane Composition | (mV) |
| PCL | −33.25 |
| PCL-collagen | −23.08 |
| PCL-collagen, crosslinked | −27.09 |
| PCL-collagen/PAH | 21.26 |
| PCL-collagen/PAH-PD | 15.42 |
| PCL-collagen/PAH-PD/PSS | −23.30 |

That which is claimed is:

1. A biocompatible, biodegradable, three-dimensional scaffold having a surface and an interior, said scaffold comprising:
a) a multiplicity of layers, wherein the layers comprise materials that degrade at different rates, with layers at the surface of the scaffold degrading prior to layers at the interior of the scaffold;
b) one or more than one first bioactive agent located at the surface of the scaffold; and
c) one or more than one second bioactive agent located at the interior of the scaffold, wherein when the scaffold is exposed to an environment surrounding the scaffold, the one or more than one first bioactive agent is released into the environment prior to release of the one or more than one second bioactive agent and following release of the one or more than one first bioactive agent and degradation of the layers at the surface of the scaffold, the one or more than one second bioactive agent is released into the environment, thereby sequentially and separately releasing the one or more than one first bioactive agent and the one or more than one second bioactive agent into the environment.

2. The scaffold of claim 1, wherein the first bioactive agent is selected from the group consisting of: an antibiotic, an antimicrobial peptide, an antimicrobial agent, an inhibitor of interleukin-1 (IL-1), an inhibitor of interleukin-6 (IL-6), an inhibitor of tumor necrosis factor alpha (TNF-α), an inhibitor of matrix metalloproteinase (MMP) 1, 2, 8 and/or 9, an inhibitor of p38 mitogen activated protein kinase (MAPK), an inhibitor of extracellular signal-related kinase (ERK) (ERK1; ERK2), SBR203580 (p38 inhibitor), PD98059 (ERK inhibitor), U0126 (inhibitor of MMP expression) simvastatin (inhibitor of MMP-1 expression), an anti-inflammatory agent and any combination thereof.

3. The scaffold of claim 1, wherein the one or more than first bioactive agent at the surface of the scaffold comprises one or more antimicrobial agents and one or more anti-inflammatory agents.

4. The scaffold of claim 3, wherein the one or more antimicrobial agents are present in one or more layers and the one or more anti-inflammatory agents are present in one or more layers, wherein the layers comprise materials that degrade at different rates, thereby sequentially and separately releasing the one or more antimicrobial agent and the one or more anti-inflammatory agent into the environment.

5. The scaffold of claim 1, wherein the second bioactive agent is selected from the group consisting of: hepatocyte growth factor (HGF) (HGF-1), stromal cell-derived factor (SDF-1), transforming growth factor beta (TGF-β; TGF-β1, TGF-β3), platelet derived growth factor (PDGF) (e.g., Becaplemiin; REGRANEX®, PDGF-BB), platelet releasate, epidermal growth factor (EGF), fibroblast growth factor (FGF) (FGF-2), granulocyte macrophage colony stimulating factor (GM-CSF), keratinocyte growth factor-2 (KGF-2), insulin-like growth factor (IGF) (IGF-I, IGF-II), bone morphogenetic protein (BMP) (BMP-2, BMP-4, BMP-5, BMP-6 and/or BMP-7 in any combination), interleukin-8 (IL-8), interleukin-10 (IL-10), insulin-like growth factor binding protein (IGFBP) (e.g., IGFBP-3; IGFBP-5), a growth factor, a small molecule (e.g., less than about 1000 Da), a regenerative agent and any combination thereof.

6. The scaffold of claim 1, wherein the layers comprise the following materials and first bioactive agents and second bioactive agents arranged in the following order from exterior to interior:
a) a surface layer comprising one or more than one first bioactive agent, a positively charged polyelectrolyte and a negatively charged polyelectrolyte;
b) a cross-linked protein;
c) one or more than one second bioactive agent and a positively or negatively charged polyelectrolyte;
d) a negatively or positively charged polyelectrolyte that has the opposite electrostatic charge of the polyelectrolyte of (c); and
e) one or more scaffolds comprising degradable synthetic polymers, degradable natural polymers and any combination thereof.

7. The scaffold of claim 6, wherein (a)-(e) comprise the following:
a) an antimicrobial agent and/or SB203580 and/or PD98059 as first bioactive agents, PAH as the positively charged polyelectrolye and PSS as the negatively charged polyelectrolyte;
b) collagen crosslinked with genipin;
c) PDGF and/or BMP2 as second bioactive agents and polycation poly(allylanion hydrochloride) (PAH) as the positively charged polyelectrolyte;
d) polyanion (polyacrylic acid) (PAA) as the negatively charged polyelectrolyte; and
e) 50:50 poly(lactic-co-glycolic acid) (PLGA):collagen as the scaffold;

8. The scaffold of claim 1, wherein the scaffold has a symmetrical organization and wherein the layers comprise the following materials and the one or more than one first bioactive agent and one or more than one second bioactive agent arranged in the following order in cross section:
a) a surface comprising one or more than one first bioactive agent, a positively charged polyelectrolyte and a negatively charged polyelectrolyte;
b) a cross-linked protein;
c) one or more than one second bioactive agent and a positively or negatively charged polyelectrolyte;
d) a negatively or positively charged polyelectrolyte that has the opposite electrostatic charge of the polyelectrolyte of (c);
e) a degradable polymer;
f) a negatively or positively charged polyelectrolye;
g) one or more than one second bioactive agent that can be the same or different from the one or more than one second bioactive agent of (c) and a positively or negatively charged polyelectrolyte that has the opposite electrostatic charge of the polyelectrolyte of (f);
h) cross-linked protein; and
i) a surface coating comprising one or more than one first bioactive agent that can be the same or different from the one or more than one first bioactive agent of (a), a positively charged polyelectrolyte and a negatively charged polyelectrolyte.

9. The scaffold of claim 8, wherein (a)-(i) comprise the following:
a) an antimicrobial agent and/or SB203580 and/or PD98059 as first bioactive agents, PAH as the positively charged polyelectrolye and PSS as the negatively charged polyelectrolyte;
b) collagen crosslinked with genipin;
c) PDGF and/or BMP2 as second bioactive agents and polycation poly(allylanion hydrochloride) (PAH) as the positively charged polyelectrolyte;
d) polyanion (polyacrylic acid) (PAA) as the negatively charged polyelectrolyte;
e) 50:50 poly(lactic-co-glycolic acid) (PLGA):collagen as the degradable polymer;
f) PAA as the negatively charged polyelectrolyte;
g) PDGF and/or BMP2 as second bioactive agents and PAH as the positively charged polyelectrolyte;
h) collagen crosslinked with genipin; and
i) an antimicrobial agent and/or SB203580 and/or PD98059 as first bioactive agents, PAH as the positively charged polyelectrolye and PSS as the negatively charged polyelectrolyte.

10. A biocompatible, biodegradable, three-dimensional scaffold having a surface and an interior, said scaffold comprising:
a) a multiplicity of layers, wherein the layers comprise materials that degrade at different rates, with layers at the surface of the scaffold degrading prior to layers at the interior of the scaffold;
b) one or more than one anti-inflammatory agent located at the surface of the scaffold; and
c) one or more than one regenerative agent located at the interior of the scaffold, wherein when the scaffold is exposed to an environment surrounding the scaffold, the anti-inflammatory agent is released into the environment prior to release of the regenerative agent and following release of the anti-inflammatory agent and degradation of the layers at the surface of the scaffold, the regenerative agent is released into the environment, thereby sequentially and separately releasing the anti-inflammatory agent and the regenerative agent into the environment.

11. The scaffold of claim 10, wherein an antimicrobial agent is located at the surface of the scaffold.

12. The scaffold of claim 11, wherein the antimicrobial agent is located in one or more than one outer layer at the surface of the scaffold and the anti-inflammatory agent is located in one or more than one inner layer at the surface of the scaffold, whereby when the scaffold is exposed to the environment surrounding the scaffold, the antimicrobial agent is released into the environment prior to release of the anti-inflammatory agent.

13. The scaffold of claim 1, wherein the layers comprise a material selected from the group consisting of: collagen, gelatin, polycation poly(allylanion hydrochloride) (PAH), polyanion (polyacrylic acid) (PAA), polycation poly(styrene sulfonate) (PSS), poly(lactic-co-glycolic acid) (PLGA), polyglycolide, poly(glycolide-co-caprolactone), poly(glycolide-co-trimethylene carbonate), polycaprolactone (PCL), polyurethane (PU), polypropylene carbonate, polyglycolic acid, polyhydroxybutyrate, polylactic acid, polydioxanone, chitosan, laminin, glycosaminoglycan, proteoglycan, heparin, elastin, fibrin, fibronectin, chondroitin sulphate proteoglycan, thiolated collagen, thiolated laminin; thiolated fibronectin, thiolated heparin, thiolated hyaluronic acid, thiolated hyaluronan-collagen-fibronectin, cellulose, hydroxyapatide, calcium phosphate and any combination thereof.

14. A method of sequentially and separately delivering an anti-inflammatory agent and then a regenerative agent to a subject having a disorder in which reduction of inflammation followed by tissue regeneration at a lesion site in the subject is indicated, comprising contacting the lesion site of the subject with the scaffold of claim 1, wherein the one or more than one first bioactive agent comprises an anti-inflammatory agent and the one or more than one second bioactive agent comprises a regenerative agent, for a period of time sufficient to deliver the anti-inflammatory agent to reduce inflammation at the lesion site and then deliver the regenerative agent to regenerate tissue at the lesion site after inflammation has been reduced.

15. The method of claim 14, further comprising sequentially and separately delivering an antimicrobial agent to the subject, comprising contacting the lesion site of the subject with the scaffold, wherein the scaffold comprises one or more than one outer layer and one or more than one inner layer at the surface of the scaffold and wherein the one or more than one first bioactive agent further comprises an anti-microbial agent located in the one or more than one outer layer and the anti-inflammatory agent is located in the one or more than one inner layer, for a period of time sufficient to deliver the antimicrobial agent to treat infection at the lesion site and then deliver the anti-inflammatory agent to reduce inflammation at the lesion site and then deliver the regenerative agent to regenerate tissue at the lesion site after inflammation has been reduced.

16. The method of claim 14, wherein the disorder is selected from the group consisting of diabetic ulcer and periodontal disease.

17. The method of claim 14, wherein the lesion site is contacted with the scaffold for a period of time sufficient to reduce inflammation by more than 50%.

18. A method of treating diabetic ulcer in a subject, comprising contacting the diabetic ulcer of the subject with an effective amount of the scaffold of claim 1.

19. A method of treating periodontal disease in a subject, comprising contacting diseased periodontal tissue of the subject with an effective amount of the scaffold of claim 1.

20. A method of enhancing tissue regeneration and/or healing at a lesion and/or wound site in a subject by first treating infection and/or reducing inflammation at the site, thereby enhancing tissue regeneration and/or healing at the site, comprising contacting the lesion and/or wound site with an effective amount of the scaffold of claim 1.
